(19) United States
(12) Patent Application Publication
(10) Pub. No.: US 2023/0285545 A1
(43) Pub. Date: Sep. 14, 2023

(54) POLYPEPTIDES, VACCINE COMPOSITIONS, AND USE THEREOF FOR INDUCING IMMUNE RESPONSE TO SARS-COV-2 IN PRIMATES

(71) Applicant: Kang ZHANG, Del Mar, CA (US)

(72) Inventors: Kang ZHANG, Del Mar, CA (US); Gen LI, Shenyang (CN); Guangxi ZANG, Shenyang (CN)

(21) Appl. No.: 18/092,544

(22) Filed: Jan. 3, 2023

Related U.S. Application Data
(63) Continuation of application No. PCT/US21/40583, filed on Jul. 6, 2021.
(60) Provisional application No. 63/048,070, filed on Jul. 3, 2020.

(51) Int. Cl. A61K 39/215; A61P 37/04

(52) U.S. Cl. CPC A61K 39/215; A61P 37/04; A61K 2039/55505

(57) ABSTRACT

Disclosed herein, in some embodiments, are methods and compositions for inducing an immune response against SARS-CoV-2 in a primate in need thereof with a recombinant polypeptide, wherein the at least a portion of the recombinant polypeptide corresponds to an amino acid residue sequence within the Receptor Binding Domain (RBD) of SARS-CoV-2 spike protein capable of forming a binding interface that interacts with a viral receptor of the primate.

### CROSS-REFERENCE TO RELATED APPLICATION

This application is a continuation of International Application No. PCT/US21/40583, filed Jul. 6, 2021, which claims priority to U.S. Provisional Application No. 63/048,070 filed Jul. 3, 2020, the disclosure of each of which is incorporated herein by reference in its entirety.

### BACKGROUND OF THE DISCLOSURE

Vaccines are useful for inducing immune response and provide acquired immunity against certain infectious diseases. Novel Coronavirus SARS-CoV-2 causes a respiratory illness called COVID-19, which was declared as a pandemic by the World Health Organization.

### SUMMARY OF THE DISCLOSURE

Disclosed herein, in some embodiments, is a method for inducing an immune response against SARS-CoV-2 in a primate in need thereof, the method comprising the step of administrating to said primate an immunogenetically effective amount of a recombinant polypeptide, wherein the at least a portion of the recombinant polypeptide corresponds to an amino acid residue sequence within the Receptor Binding Domain (RBD) of SARS-CoV-2 spike protein capable of forming a binding interface that interacts with a viral receptor of the primate, wherein the effective amount of the recombinant polypeptide binds to the viral receptor of the primate to induce an immune response against SARS-CoV-2 in the primate.

Disclosed herein, in some embodiments, is a method for preventing, inhibiting, reducing, eliminating, protecting or delaying the onset of an infection or an infectious clinical condition caused by SARS-CoV-2 in a primate in need thereof, the method comprising the step of administrating to said primate a therapeutically effective amount of a recombinant polypeptide, wherein the at least a portion of the recombinant polypeptide corresponds to an amino acid residue sequence within the Receptor Binding Domain (RBD) of SARS-CoV-2 spike protein capable of forming a binding interface that interacts with a viral receptor of the primate, wherein the effective amount of the recombinant polypeptide binds to the viral receptor of the primate to induce an immune response against SARS-CoV-2 in the primate.

In some embodiments, the infectious clinical condition is COVID-19.

In some embodiments, the amino acid residue within RBD of SARS-CoV-2 spike protein is amino acid residue 319-545. In some embodiments, the recombinant polypeptide comprises or consists essentially of a subsequence within the amino acid residue 319-545 of SARS-CoV-2 spike protein with up to 5 amino acids removed from either end or both ends of amino acid residue 319-545.

In some embodiments, the recombinant polypeptide consists essentially of amino acid residue 319-545 within the RBD of SARS-CoV-2 spike protein.

In some embodiments, said SARS-CoV-2 spike protein is a SARS-CoV-2 S1 protein.

In some embodiments, the recombinant polypeptide has an average molecular weight of more than 28 kDa.

In some embodiments, the recombinant polypeptide has an average molecular weight of from about 28 kDa to about 40 kDa.

In some embodiments, the recombinant polypeptide has an average molecular weight of about 34 kDa.

In some embodiments, the recombinant polypeptide comprises a plurality of N-glycosylation sites.

In some embodiments, the recombinant polypeptide comprises 17 glycan moieties on N331.

In some embodiments, the recombinant polypeptide comprises 12 glycan moieties on N334.

In some embodiments, the recombinant polypeptide comprises 19 glycan moieties on N343.

In some embodiments, the recombinant polypeptide comprises a plurality of O-glycosylation sites.

In some embodiments, the O-glycosylation sites comprise seven serine residues (S366, S371, S373, S375, S438, S443 and S514).

In some embodiments, the O-glycosylation sites comprise three threonine residues (T333, T376 and T523).

In some embodiments, the method further comprises co-administering to the primate an immunologic adjuvant. In some embodiments, the immunologic adjuvant is selected from the group consisting of aluminum salts, Toll-Like-Receptor (TLR) agonist, oil-in-water emulsion adjuvants, saponin-based adjuvants, and combination thereof.

In some embodiments, the immunologic adjuvant is aluminum hydroxide or a hydrate thereof.

In some embodiments, the immune response against SARS-CoV-2 induced by the effective amount of the recombinant polypeptide blocks SARS-CoV-2 infection in the primate by at least about 60%, at least about 65%, at least about 70%, at least about 75%, at least about 80%, at least about 85%, at least 90%, at least 95%, at least 96%, at least about 97%, at least about 98%, or at least 99%.

In some embodiments, the immune response against SARS-CoV-2 induced by the effective amount of the recombinant polypeptide blocks SARS-CoV-2 infection in the primate by at least 95%.

In some embodiments, the immune response against SARS-CoV-2 induced by the effective amount of the recombinant polypeptide blocks SARS-CoV-2 infection in the primate by at least 99%.

Disclosed herein, in some embodiments, is a composition for inducing an immune response against SARS-CoV-2 in a subject in need thereof, the composition comprising a recombinant polypeptide and an immunologic adjuvant, wherein the at least a portion of the recombinant polypeptide corresponds to an amino acid residue within the Receptor Binding Domain (RBD) of SARS-CoV-2 spike protein capable of forming a binding interface that interacts with a viral receptor of the subject.

In some embodiments, the immunologic adjuvant is selected from the group consisting of aluminum salts, Toll-Like-Receptor (TLR) agonist, oil-in-water emulsion adjuvants, saponin-based adjuvants, and combination thereof.

In some embodiments, the immunologic adjuvant is aluminum hydroxide or a hydrate thereof.

In some embodiments, the immunologic adjuvant is a TLR agonist selected from the group consisting of TLR2 agonist, TLR3 agonist, TLR4 agonist, TLR5 agonist, TLR7 agonist, TLR8 agonist, TLR9 agonist, and combinations thereof.

In some embodiments, the immunologic adjuvant is a TLR7/8 agonist.

In some embodiments, the TLR7/8 agonist is imiquimod or resiquimod.

In some embodiments, the immunologic adjuvant is an oil-in-water emulsion adjuvant comprising squalene.

In some embodiments, the composition further comprises at least one pharmaceutically acceptable carriers, excipients, auxiliaries, and/or diluents.

In some embodiments, the amino acid residue within RBD of SARS-CoV-2 spike protein is amino acid residue 319-545.

In some embodiments, the recombinant polypeptide comprises or consists essentially of amino acid residue 319-545 within the RBD of SARS-CoV-2 spike protein.

In some embodiments, said SARS-CoV-2 spike protein is a SARS-CoV-2 S1 protein.

In some embodiments, the recombinant polypeptide has an average molecular weight of more than 28 kDa.

In some embodiments, the recombinant polypeptide has an average molecular weight of from about 28 kDa to about 40 kDa.

In some embodiments, the recombinant polypeptide has an average molecular weight of about 34 kDa.

In some embodiments, the recombinant polypeptide comprises a plurality of N-glycosylation sites.

In some embodiments, the recombinant polypeptide comprises 17 glycan moieties on N331.

In some embodiments, the recombinant polypeptide comprises 12 glycan moieties on N334.

In some embodiments, the recombinant polypeptide comprises 19 glycan moieties on N343.

In some embodiments, the recombinant polypeptide comprises a plurality of O-glycosylation sites.

In some embodiments, the O-glycosylation sites comprise seven serine residues (S366, S371, S373, S375, S438, S443 and S514).

In some embodiments, the O-glycosylation sites comprise three threonine residues (T333, T376 and T523).

### BRIEF DESCRIPTION OF THE DRAWINGS

Various aspects of the disclosure are set forth with particularity in the appended claims. The file of this patent contains at least one drawing/photograph executed in color. Copies of this patent with color drawing(s)/photograph(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIG. 1. Characterization of the SARS-CoV-2 S RBD protein

a. A schematic view of the SARS-CoV-2 S protein. The indicated domains and elements, including signal peptide (SP), N-terminal domain (NTD), receptor binding domain (RBD), heptad repeat 1 and 2 (HR1 and HR2), transmembrane domain (TM) and cytoplasmic domain (CP), are marked. For S RBD preparation, the RBD region was engineered to include an N-terminal GP67 signal peptide. b. A representative elution chromatograph of the recombinant RBD protein on a Superdex 200 increase column. The inset figures showed the SD S-PAGE and western-blotting analyses of the eluted RBD samples. c. Glycosylated peptides in RBD identified by mass spectrometry. The N-glycosylation and O-glycosylation sites are marked with the residue numbers. d. The abundance of glycosylation. The number of MS/MS spectra of each glycosylated peptides listed in c and their corresponding unmodified peptides were counted. Nine sample preparation and MS acquisition methods are applied. Each spot means the results from one method. e. An overview of the glycosylation sites illustrated based on the solved complex structure of SARS-CoV-2 RBD bound to ACE2 (PDB code: 6LZG). The identified sites, colored red for O-glycosylation and blue for N-glycosylation, are shown as spheres and labeled. The approximate boundary between the core and external subdomains in RBD are marked with a dashed line. The right panel (surface representation) was generated by rotating the structure in the Left panel (cartoon representation) around a vertical axis for about 90°. f. The real-time binding profile between our purified RBD protein and ACE2 characterized by SPR Biacore.

FIG. 2. Serum antibody response against S protein RBD in patients and in mice as determined by ELISA.

a. The mice were immunized with 5 μg recombinant RBD protein per mouse in 50 μl in the presence of aluminum hydroxide, compared with the control groups including recombinant RBD protein alone, with aluminum hydroxide (AL), pre-immune or PBS alone. Sera were collected from the mice 7 days after the first dose of vaccine and were tested with different dilution for IgG and IgM against recombinant RBD protein using ELISA as described in Methods. Data are presented as mean±SEM of five mouse sera in each group. P-values were determined by two-way ANOVA. P-values indicated RBD+AL vs AL or PBS or pre-immune groups. RBD+AL vs RBD alone in IgG level: P<0.0001 and P<0.002 at dilution of 1:25 and 1:50 respectively. AL: Al(OH)3, RBD: recombinant RBD protein, RBD+AL: the addition of the recombinant RBD protein to Al(OH)3, PBS—phosphate buffered saline. Similar results were repeated in three independent experiments. b. The mice were immunized with 0.1-10 μg recombinant RBD protein per mouse in 50 μl in the presence of AL, or AL alone or PBS alone. Sera were collected from the mice 7 days after the first dose of vaccine and were tested at 1:50 dilution for IgG and IgM against S protein RBD using ELISA. Data were presented as mean±SEM of six mouse serum samples in each group. P-values were determined by one-way ANOVA. c. Sera were collected 14 days after two vaccinations on day 0 and day 7 with 5 μg recombinant RBD protein per mouse in 50 μl in the presence of aluminum hydroxide, or recombinant RBD protein alone, and the level of the IgG of the sera was tested at the different dilution by ELISA. Similar results were repeated in three independent experiments. d. Sera were collected from 9 days after the three vaccinations on day 0, day 14 and day 21 with the same doses as c. e. Serum samples were collected from 16 patients infected with SARS-CoV-2 (and recovered) and 20 healthy donors and detected with 1:5 diluted sera with ELISA as described in Methods. Data are presented as the mean±SEM. P-values were determined by one-way ANOVA.

FIG. 3. Functional characterization of the sera from the immunized monkeys and the protection of the Non-human primates, Macaca mulatta, from SARS-CoV-2 infection.

a. Inhibition of the S protein RBD binding to cell-surface ACE2. Recombinant SARS-CoV-2 RBD-Fc fusion protein was added to ACE2-positive Huh7 cells to a final concentration of 0.1 μg/ml in the presence or absence of the sera at a dilution of 1:5, followed by incubating with anti-human IgG-FITC conjugate. The binding assay of RBD-Fc with ACE2 was performed by flow cytometry as described in Methods labelled as Sera/RBD (stained with sera pooled from 5 mice immunized with RBD vaccine day 7 after the first vaccination), Sera/PBS (stained with sera from the mice treated by PBS as a control), Positive control (without the presence of Sera/RBD or Sera/PBS), Negative control (the cells stained with anti-human IgG-FITC conjugate alone) (n=3 independent experiments). b. The neutralization of SARS-CoV-2 pseudovirus infection by the sera. Supernatants containing SARS-CoV-2 pseudovirus were preincubated with mouse sera which was serially diluted 2-fold. After incubated for 1 hour at 37° C., the mixture was added to ACE2-transfected 293T (293T/ACE2) cells to detect viral infectivity. The number of green fluorescent protein (GFP) expression in the infected cell was determined by fluorescent microscopy and flow cytometry. Sera/RBD (sera pooled from 5 mice immunized with RBD vaccine day 14 after the first vaccination) and Sera/PBS (sera from the mice treated by PBS as a control), Untreated (infection with SARS-CoV-2 pseudovirus without sera). Similar results were repeated in three independent experiments. c-g. Non-human primates (Macaca mulatta) were immunized with two injections on day 0, day 7 via the intramuscular route with 20 μg or 40 μg per dose and then challenged with SARS-CoV-2 intranasally (0.5 ml, 106 pfu/ml) on day 28 after the first vaccination as described in methods. c. Neutralization of live SARS-CoV-2 infection by the immune sera from non-human primates. SARS-CoV-2 was added to monolayers of Vero E6 in the presence of immune sera from non-human primates in a series of 2-fold dilutions. Cytopathic effect (CPE) induced by SARS-CoV-2 infection was recorded under microscope and the virus-neutralizing titers was determined. A quantitative real-time reverse transcription-PCR (qRT-PCR) was employed to measure viral genomic RNA (gRNA) and viral subgenomic RNA (sgRNA, indicative of virus replication) in lung tissues (d), the throat swabs (e), and the anal swabs (f). Pictured are individual values with geometric mean bars. 40 μg RBD+AL: vaccinated with 40 μg recombinant RBD protein with Al(OH)₃ (n=4); 20 μg RBD+AL: vaccinated with 20 μg recombinant RBD protein to Al(OH)₃ (n=3); Untreated: treated with PBS (control, n=3), AL: Al(OH)₃ alone (adjuvant control, n=2). g. Histopathological changes in lung tissues observed by light microscopy. Normal histology in the two vaccinated groups and severe pneumonia in the two control groups.

FIG. 4. The pathways involved with the recombinant RBD stimulation and cellular immune response.

a. Wild-type C57BL/6 mice and mice deficient in Cd4−/−, Cd8a−/−, Tlr2−/−, Tlr4−/−, Sting1−/−, Casp1−/−, Nlrp3−/−, and Il-1β−/− were immunized with the recombinant RBD protein (5 μg per mice) and sera were collected on day 7 after the first dose of vaccine and were tested for the antibody against RBD at the dilution of 1:25. The data are expressed as mean±SEM. P-values were determined by two-way ANOVA (4-6 mice per group). Similar results were repeated in two independent experiments. b. Cytokines produced by the spleen lymphocytes were detected by ELISA under the stimulation of the recombinant RBD. Mice immunized with the candidate RBD vaccine or treated with PBS were sacrificed 7 days after the first dose of vaccine to isolate lymphocytes which were then stimulated with recombinant RBD for three days, and the supernatants were collected for the level of secreted IL-4 and IFN-γ by ELISA assays, as described in Methods. The data are expressed as mean±SD. P-values were determined by unpaired Student's t tests (5 mice per group). Similar results were repeated in three independent experiments. c. The lymphocytes in the spleen were collected from the mice with the recombinant RBD (5 μg per mice) 7 days after the first vaccination and were incubated with RBD for three days, and then RBD-reactive memory CD4 or CD8 was analyzed by flow cytometry by gating CD4+ or CD8+CD44high+B220−MHCII-IFN-γ+ or IL-4+, as described in Method. Similar results were repeated in three independent experiments. d. Sera were collected from the mice 7 days after the first dose of vaccine and plasma level of cytokines such as TNF-α, IFN-g, IFN-α, IFN-b, IL-6, IL-4 were measured by ELISA. AL: Al(OH)3, RBD: recombinant RBD, RBD+AL: the addition of the recombinant RBD to Al(OH)3.

FIG. 5. Identification of serum antibody against S protein RBD and neutralizing antibody against SARS-CoV-2 pseudovirus in rabbits. a. The rabbits were immunized with 1-40 μg recombinant RBD protein per rabbit in 500 μl in the presence of aluminum hydroxide [Al(OH)₃], or Al(OH)₃ or PBS alone. Rabbit were immunized with three vaccinations on day 0, day 14 and day 21, collected sera 7 days after each boost. Sera were collected from the rabbits 7 days after the third vaccination and were tested at different dilution for IgG against S protein RBD using ELISA. Data are presented as the mean±SEM of six rabbits' sera in each group. AL: Al(OH)₃, RBD: recombinant RBD protein, RBD+AL: the addition of the recombinant RBD protein to Al(OH)₃. b. Potent neutralization of SARS-CoV-2 pseudovirus infection by the sera from the rabbit immunized with recombinant RBD vaccine. Infection of HEK293 cells expressing human ACE2 by SARS-CoV-2 pseudovirus was determined in the presence of rabbit sera at a series of 3-fold dilutions. Percentage of neutralization was presented as mean±SEM. The sera from the rabbits immunized 7 days after the third vaccination with the dose of 20 μg recombinant RBD protein per rabbit in 500 μl in the presence of aluminum hydroxide in the same as a. The neutralization assay of SARS-CoV-2 pseudovirus was performed as described in Methods.

FIG. 6. Identification of serum antibody against S protein RBD and in non-human primates. The non-human primates (Macaca mulatta) were immunized with 40 μg recombinant RBD protein per monkey in 1 ml in the presence of aluminum hydroxide on day 0 and day 7, and sera were obtained at 7 day (a) and 14 days (b) after the first vaccination or before the vaccination (Pre-immune). Also, the monkeys were treated with PBS as a control. Sera were tested at the different dilution for IgG against recombinant RBD protein using ELISA was as described in Methods. Data are presented as the mean±SEM of 10 monkey sera in each group. P-values were determined by two-way ANOVA. P-values indicated RBD+AL vs PBS groups in IgG level. (c) The neutralization of the infection of SARS-CoV-2 pseudovirus by the sera from the non-human primates. The neutralization assays were performed with the sera from monkeys 14 days after the first vaccination as in b. 50% neutralization (EC50) was presented as mean±SEM. The neutralization assay of SARS-CoV-2 pseudovirus was described in Methods.

FIG. 7. The neutralization of the infection of SARS-CoV-2 pseudovirus by the sera from mice or rabbits. a. Supernatants containing SARS-CoV-2 pseudovirus were preincubated with the sera from mice which was serially diluted 2-fold. After incubated for 1 hour at 37° C., the mixture was added to ACE2-transfected 293T (293T/ACE2) cells to detect viral infectivity. The number of green fluorescent protein (GFP) expression in the infected cell was determined by fluorescent microscopy and flowcytometry. Sera/RBD (sera pooled from 5 mice immunized with RBD vaccine day 14 after the first vaccination) and Sera/PBS (sera from the mice treated by PBS as a control), Untreated (infection with SARS-CoV-2 pseudovirus without sera). b. The neutralization of the infection of SARS-CoV-2 pseudovirus was performed using the sera from rabbits 14 days after the first vaccination in the same way as a.

FIG. 8. Detection of the neutralizing antibodies in sera of the mice vaccinated with recombinant RBD protein or the other domains of S protein. Infection of HEK293 cells expressing human ACE2 by SARS-CoV-2 pseudovirus was determined in the presence of mice sera at a series of 3-fold dilutions. 50% neutralization was presented as mean±SEM. The mice were immunized with 5 μg recombinant RBD Protein (RBD, aa 319-545), the extracellular domain protein (ECD, aa 16-1213), S1-subunit protein (S1, aa 16-685) or S2-subunit protein (S2, aa, 686-1213) in the presence of aluminum hydroxide gel on day 0, day 14 and day 21. Sera were collected from the mice after the third vaccination. All these proteins are prepared from the insect cells, as described in Methods.

FIG. 9. The induction of the neutralizing antibodies against live SARS-COV-2 in the transgenic hACE2 mice and the wild-type mice. The transgenic hACE2 mice and the wild-type mice were immunized with 10 μg recombinant RBD protein per mouse in 50 μl in the presence of aluminum hydroxide, compared with the treatment with PBS alone. Sera were collected from the mice 14 days after the second vaccination. To assess the neutralization of SARS-COV-2 infection, Vero E6 cells (5×10⁴) were pre-load in 96-well plates and grown overnight. One hundred TCID50 (50% tissue-culture infectious dose) of SARS-CoV-2 was preincubated with an equal volume of diluted sera before addition to cells. After incubation at 37° C. for 1 h, the mixture was added to Vero E6 cells. The cytopathic effect (CPE) was recorded under microscope and the neutralizing titers of the dilutions of sera resulting in complete inhibition were calculated.

FIG. 10. Adoptive therapy of splenic T cells versus immune sera from the vaccinated mice. a. hACE2 mice with C57BL/6 background received 5×10⁷ splenic T cells isolated from the mice with same C57BL/6 background 9 days after the third dose of the candidate vaccine or from the mice treated with PBS as a control. The mice were sacrificed 5 days after the challenge with live virus, and viral replication in lung tissues, lung histopathological changes, and body weight change were evaluated. The adoptive therapy based on immune sera was performed using 0.1 ml of the pooled sera from the immunized mice at the same time. b. hACE2 mice with ICR background received 0.8 ml sera from the mice 7 days after a single dose of the vaccine and challenged with live SARS-CoV-2.

### DETAILED DESCRIPTION OF THE DISCLOSURE

The outbreak of the 2019 novel coronavirus SRAS-CoV-2 has led to more than 1.5 million confirmed infections globally and the associated disease COVID-19, has claimed the life of more than 110,000 people globally as of 12 Apr. 2020^{3,4}. In an early report, the epidemic doubled in the number of infected subjects every 7.4 day³. This infection has a mean incubation period of 5.2 days and causes fever, cough, and other flu-like symptoms. Many infected patients develop pneumonia and a proportion of these patients will rapidly progress into acute respiratory failure with a very poor prognosis and high mortality^{5,6}. Person-to-person transmission has been documented⁷⁻¹⁰, and the WHO has declared SARS-CoV-2 infection as a pandemic in March 2020. Early estimates suggested that up to 60% of patients may die once they progress into the severe/critical illness stage as designated by the need of ventilators or admission into the intensive care unit^{5,6}.

The present disclosure recognizes the urgent need for an effective preventive vaccine. SARS-CoV-2 binds to cells utilizing the human angiotensin-converting enzyme 2 (ACE2) as a receptor^{1,2}. Based on our knowledge of the viral envelope protein, we hypothesized that the receptor binding domain (RBD) will be a good immunogen than the entire extra-cellular portion of the protein or the S1 and S2 domains. Note that the full-length S protein contains a large hydrophobic domain, which will render the full-length protein insoluble and impossible to purify and therefore, will not be a good vaccine candidate. Moreover, it has been reported that the full-length spike protein of SARS virus could induce antibody-dependent enhancement that will enhance lung damage in animals challenged with SARS^{11,12}.

The baculovirus expression system was chosen to express the various protein for our study as this is a commercially feasible system and can manufacture the candidate vaccine, if successful, in a commercial scale, and the vaccine generated are in general with correctly folded protein conformation^{13,14}. In fact, this technology was used in several commonly used vaccine products, including some of the cervical cancer vaccine and influenza vaccines currently in the European and the United States market^{13,14} The objective of this study was to evaluate the potential of a candidate vaccine based on the RBD domain of SARS-CoV-2, evaluate the appropriate dosing regime and testing its effect in generating neutralizing activity against SARS-CoV-2 in the recipient animals, and determine the immune pathways involved in the generation of the immune response, so as to provide the groundwork for the design of an effective SARS-CoV-2 preventive vaccine.

Provided herein are methods, systems and compositions for overcoming the above challenges with cellular reprogramming which switches a cell type that is sensitive to a mutation to a functionally related cell type that is resistant to the same mutation, therefore preserve the tissue and function. These approaches are based on the premise that 1) a mutation usually causes its detrimental effect in only a particular cell type; 2) a combination of factors enables determination of a cellular fate, and 3) there is developmental plasticity that allows for direct conversion in vivo between closely related, terminally differentiated mature cell types such as pancreas, cardiac and neural cells. Furthermore, distantly related cells can also be directly converted in vivo by appropriate combinations of developmentally relevant factors.

Methods disclosed herein may utilize a homology-independent targeted integration (HITI) strategy, based on clustered regularly interspaced short palindromic repeat-Cas9 (CRISPR-Cas9). These methods may provide efficient targeted knock-in in both dividing and non-dividing cells. These methods may be performed in vitro and in vivo. These methods may provide for on-target transgene insertion in post-mitotic cells, e.g., cells of the eye, in postnatal mammals.

### Certain General Terminologies

Unless defined otherwise, all technical and scientific terms used herein have the same meaning as is commonly understood by one of skill in the art to which the claimed subject matter belongs. It is to be understood that the foregoing general description and the following examples are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. For example, “about 5 μL” means “about 5 μL” and also “5 μL” Generally, the term “about” includes an amount that would be expected to be within experimental error. The term “about” includes values that are within 10% less to 10% greater of the value provided. For example, “about 50%” means “between 45% and 55%.” Also, by way of example, “about 30” means “between 27 and 33.”

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one antibody of the present disclosure is administered.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value. A p-value of less than 0.05 is considered statistically significant.

As used herein, the term “treating” and “treatment” refers to administering to a subject an effective amount of a composition so that the subject as a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Alternatively, treatment is “effective” if the progression of a disease is reduced or halted. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

### Polypeptides

The present disclosure contends with development of specific and effective vaccines and vaccine-based therapeutic applications targeting viral pathogens, for example, CoVs that may infect any mammalian or avian subjects. Non-limiting examples for such CoVs relate to MERS CoV and SARS-CoV-2 in particular. The vaccines are based on exact reconstitution of the functional binding-motif of the CoVs Spike protein (S1) and generation of recombinant peptides characterized by high affinity and specificity to CoVs receptors and neutralizing antibodies.

The present disclosure is based on the rationale that as the viral S1 is responsible for the host tropism and for immunogenicity in the host, it would be possible to reconstitute the functional part of the viral S1, namely the Receptor Binding Domain (RBD) or more specifically, the Receptor Binding Motif (RBM), that is capable of both of these functions, thereby targeting viral infections most effectively.

An amino acid sequence of the spike protein of SARS-CoV-2 is set forth in SEQ ID NO:1. An amino acid sequence of the spike protein of MERS-CoV is set forth in SEQ ID NO:2.

Validity of the proposed approach has been presently exemplified by successful reconstitution of the SARS-CoV-2 RBM as an independently-folding, functional binding-domain of S1, and expression of about 40-amino acid long RBM-derived peptides in E. coli, which was shown to bind both ligands: the viral receptor ACE2 and the neutralizing mAb 80R. The products produced by the technology of the disclosure serve as an immunogens and vaccines for human and/or veterinary use.

According to a first aspect, the disclosure relates to an isolated recombinant polypeptide comprising an amino acid sequence of at least one reconstituted Receptor Binding Motif (RBM) of a viral Spike protein, or of any fragments thereof. More specifically, the reconstituted RBM of the disclosure may comprise at least one linker and at least one fragment of the native RBM. In some specific embodiments, the native RBM is a to 200 amino acid sequence comprised within the Receptor Binding Domain (RBD) of said Spike protein forming a binding interface that interacts with the viral receptor. It should be further noted that in some embodiments, the native RBM is an extended 30 to 100 or more amino acid excursion, juxtaposed along the edge of the core of the RBD and tacked to the core via a tacking segment. In some specific embodiments, the reconstituted RBM comprises at least one exogenous linker. More specifically, at least one of said linker/s may replace at least one of: the tacking segment, any part or amino acid residue/s thereof and any RBM fragment or amino acid residue/s. In some embodiments, the linker/s may replace at least one amino acid residues not directly involved or participate in receptor and/or neutralizing antibodies (nAb/s) binding. In yet some further alternative embodiments, residues that may function as “contact residues” for the receptor and/or nAbs, may be replaced by at least one linker/s. In yet some further embodiments, at least one residue involved directly or indirectly in receptor and/or nAb/s binding, may be replaced by said linkers.

The polypeptide of the disclosure is derived from a particular region of the receptor binding domain of a spike protein, or any other peplomer, that interacts with the viral receptor on the target cell. A “peplomer”, as used herein, a glycoprotein structural unit found on viral capsid or the lipoprotein envelope of enveloped viruses, e.g. H and N spikes of influenza virus. The peplomers are essential for both host specificity and viral infectivity. The term “peplomer” or “spike” is typically used to refer to a grouping of heterologous proteins on the virus surface that function together. The term receptor binding domain (RBD) as used herein refers to a region, segment or domain within a polypeptide studding (or covering) the envelope of a virus that is associated with or mediates the binding of the virus to a host cell, in particular to a host cell receptor. Polypeptides studding the envelope of a virus are commonly referred to as spikes or spike proteins. Particular RBD encompassed by the present disclosure are of the spike proteins of SARS-CoV-2.

The recombinant polypeptides may comprise at least one amino acid residue derived from the RBD of the CoV S1 proteins specified above.

The term receptor binding motif (RBM) as used herein refers to a region within the RBD that is in direct contact with the host cell or the viral receptor on the host cell. In other words the term refers to a region within a polypeptide studding (or covering) the envelope of a virus, specifically within the RBD thereof, that mediates binding between the virus and host cell though non-covalent interactions. For example and as described herein, the co-crystallization of the SARS-CoV-2 RBD bound to its receptor ACE2 revealed that the actual binding interface lies within an extended excursion juxtaposed along the edge of the core of the RBD and constitutes the Receptor Binding Motif. The RBM may be identified by any method known in the art based on the interaction formed between the spike protein and a host cell. In certain embodiments, the RBM of the viral spike protein may be an extended 30, 31, 32, 33, 34, 45, 46, 37, 38, 39, 40, 41, 42, 43, 44, 4546, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 110, 120, 130, 140, 150, 160, 170, 180, 190, 200 or more amino acid excursion. As noted above, at least part of the RBM participates in receptor interaction.

As indicated above, the disclosure provides reconstituted RBMs that comprise at least one linker and at least one fragment derived from a native RBM. Reconstituted RBM, as used herein refers to a recombinant or synthetically non-natural RBM, build up again from parts, reconstructed, recombined and recomposed of fragments of the native RBM, that may in certain embodiments attached or linked together by at least one linker. As noted above, the reconstituted RBM of the disclosure comprise at least one fragment or amino acid residue or sequence of the native or natural RBM, and at least one exogenous linker.

Specific embodiments for the reconstituted RBMs provided by the disclosure are described in more detail herein after. Native protein as used herein refers to a protein in its properly folded and/or assembled form, which is operative and functional. The native state of a protein may possess all four levels of bio-molecular structure, with the secondary through quaternary structure being formed from weak interactions along the covalently-bonded backbone. In still further embodiments, this term relates to the RBM of the natural S1 protein as appropriately expressed and presented in the natural viral envelop or capsid.

More specific embodiments refer to RBM of the S1 protein of different CoVs, specifically, the RBMs of S1 proteins of SARS-CoV-2. In yet some further particular non limiting embodiments, this term refers to the RBMs derived from S1 proteins of SARS-CoV-2.

Thus, in some embodiments, the reconstituted RBMs of the disclosure may comprise the sequence of the native RBM, were residues, fragments or segments that are not directly involved or participate in receptor interaction or receptor contact may be replaced by at least one linker. It should be however understood that the reconstituted RBMs of the disclosure may comprise at least part, and preferably, most or all residues that participate in interaction with the receptor. In yet some further alternative embodiments the linker/s of the reconstituted RBMs of the disclosure may replace at least one amino acid residues involved directly or indirectly in receptor and/or nAb/s binding. Nevertheless, it should be understood that in some embodiments, the reconstituted RBMs of the disclosure may comprise at least one or more, specifically, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 2, at least 23, at least 24, at least 25, at least 26, at least 27, at least 28, at least 29 at least 30 or more amino acid RBM amino acid residues that are directly or indirectly involved in in receptor and/or nAb/s binding.

In this connection, it should be noted that in certain embodiments, amino acid sequences or amino acid residues that are not directly or indirectly involved in interaction with various neutralizing antibodies, may be also replaced, removed, excluded or substituted by at least one linker.

The reconstituted RBMs of the disclosure may comprise the amino acid sequence of the native RBM, or of any fragment thereof “Fragment” with respect to polypeptide sequences means polypeptides that comprise at least 10%, at least 20%, at least 30%, at least 40%, at least 50%, at least 60%, at least 70%, at least 80%, at least 90% or at least 95% of the complete segment of the native RMB of said viral spike protein, specifically the CoV S1. In some embodiments, fragments of the RBMs may comprise at least 5, at least 10, at least 15, at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more and at least 100 amino acids or more of said spike RBMs.

As indicated above, the reconstituted RBM of the disclosure comprises at least one linker that replaces in some embodiments, the tacking segment of the native RBM, or any part or amino acid residue/s thereof. As such, in some embodiments the reconstituted RBM of the disclosure lacks at least part of the native tacking segment. In further embodiments, the reconstituted RBM of the disclosure may comprise more than one linker, for example, 2, 3, 5, 6, 7, 8, 9, 10 or more that replace at least part of the tacking segment, or a sequence that comprise at least part of the tacking segment. It should be further appreciated that in some particular embodiments, in addition to linker/s that replace the tacking segment, the reconstituted RBM of the disclosure may further comprise at least one linker that replace/s at least one amino acid residue/s located in other segments of the native RBM. In yet some further embodiments, the reconstituted RBM polypeptide of the disclosure may comprise at least one linker that replaces at least one amino acid residue of the RBM, or any fragments thereof not directly involved in reception and/or nAb/s binding. Alternatively, the linker/s may replace at least one amino acid residue of the RBM directly or indirectly involved and participate in reception and/or nAb/s binding.

Still further, the reconstitute RBM polypeptides of the disclosure may comprise between about 10 to 100 amino acid residues, specifically, between about 20 to 50 amino acid residues. Specifically, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100 or more amino acid residues.

In some specific embodiments, the reconstitute RBM polypeptides of the disclosure may comprise at least one amino acid sequence derived from the RBM of a Coronavirus (CoV) S1 protein.

Thus, the main goal of the present disclosure is to provide means to combat Coronaviruses (CoVs) infections and to prevent spread of such infections via other animals to humans and/or to domestic animals as well as to prevent human to human infections.

CoVs are common in humans and usually cause mild to moderate upper-respiratory tract illnesses. There are four main sub-groupings of coronaviruses, known as alpha, beta, gamma, and delta.

Coronaviruses are species in the genera of virus belonging to one of two subfamilies Coronavirinae and Torovirinae in the family Coronaviridae, in the order Nidovirales. Herein this term refers to the entire family of Coronavirinae (in the order Nidovirales). Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large surface projections creating an image reminiscent of a crown. This morphology is created by the viral spike (S) peplomers, which are proteins that populate the surface of the virus and determine host tropism.

There are many CoVs that naturally infect animals, the majority of these usually infect only one animal species or, at most, a small number of closely related species, but not humans. CoV strains that are particular subject of the present disclosure, due to their extreme virulence and hazard in humans, are those that have been transmitted from animals to humans. For example, SARS-CoV-2 can infect people and animals, including monkeys, Himalayan palm civets, raccoon dogs, cats, dogs, and rodents. Therefore, in certain embodiments, the Coronavirus may be a CoV infecting mammalian subject/s, avian subject/s or any other vertebrates.
Thus the term Coronaviruses (designated herein CoVs) for the purposes of the present disclosure encompasses four main sub-groupings of coronaviruses, known as Alpha, Beta, Gamma, and Delta.
More specifically, under this term is meant the enveloped viruses with a positive-sense RNA genome (ssRNA+) and with a nucleocapsid of helical symmetry; and also large RNA viruses with the genomic size of ranges from approximately 26 to 32 kilobases; and further viruses with the characteristic morphology of large, bulbous surface projections under electron microscopy, which is created by the viral spike (S) peplomers, i.e. viral surface proteins determining host tropism and immunogenicity.
The present disclosure in particular concerns the two novel coronaviruses that have recently emerged: SARS-CoV-2. There are currently no clinically approved vaccines or antiviral drugs available for either of these infections; thus, the development of effective therapeutic and preventive strategies that can be readily applied to new emergent strains is a research priority.
In the specific case of the SARS-CoV-2, a defined receptor-binding domain on S mediates the attachment of the virus to its cellular receptor, angiotensin-converting enzyme 2 (ACE2) (see below). Some CoVs, specifically the members of Beta CoV subgroup A, also have a shorter spike-like protein called hemagglutinin esterase (HE).

Within the above group of human CoVs. of particular relevance to the present disclosure are the SARS-CoV-2 associated with Severe Acute Respiratory Syndrome, and the MERS CoV associated with Middle East Respiratory Syndrome, as for being the primary causes of life-threatening infectious diseases and epidemics in humans.

The other human CoVs are believed to cause a significant percentage of all common colds in human adults (primarily in the winter and early spring seasons). In certain individuals CoVs may further be a direct or indirect cause of pneumonia, i.e. direct viral pneumonia or a secondary bacterial pneumonia.

As indicated above, in some particular embodiments, the reconstituted RBM of the disclosure may comprise at least one linker that replaces the loop tacking segment or any part thereof and optionally at least one further linker that replaces at least one amino acid residue located in other parts of the RBM. In certain embodiments, such additional linker/s may replace for example Cys503. In more specific embodiments, such additional linker may be an amino acid linker that replaces Cys503 with any other amino acid residue, for example. Ser or Gly.

In some alternative embodiments, the linker may replace residues other than Cys503, and therefore retain the Cys503.

In yet some further embodiments, the reconstituted RBM may comprise at least one linker that may replace or may be added to other parts or fragments of the native RBM. More specifically, at least one of the linker/s may replace at least one RBM fragment or amino acid residue/s not directly involved in receptor binding.

In yet some alternative embodiments, the linker comprised within the reconstituted RBM polypeptide of the disclosure may replace at least one RBM fragment or amino acid residue directly or indirectly involved in receptor and/or nAb/s binding.

It should be appreciated however, that at least some of the reconstituted RBM polypeptides of the disclosure comprise at least one or more amino acid residue/s directly or indirectly involved in receptor and/or nAb/s binding.

Specifically SARS-CoV-2 has been associated with a viral disorder characterized by high fever, dry cough, shortness of breath (dyspnea) or breathing difficulties, and atypical pneumonia. The complete SARS-CoV-2 has been analyzed and published. Since then a large number of SARS strains have been isolated and characterized, and are accessible via the Centers for Disease Control and Prevention (CDC), or the National Center for Biotechnology Information, e.g. the sequence of the SARS-CoV-2.

More specially, SARS-CoV- is a coronavirus that causes COVID-19. SARS-CoV-2 is a positive and single stranded RNA virus belonging to a family of enveloped coronaviruses. Its genome is about 29.7 kb. The SARS virus has 13 known genes and 14 known proteins. SARS is similar to other coronaviruses in that its genome expression starts with translation of two large ORFs, 1a and 1b, both of which are polyproteins. The functions of several of these proteins are known, ORFs 1a and 1b encode the replicase and there are four major structural proteins: nucleocapsid, spike, membrane and envelope. It also encodes for eight unique proteins, known as the accessory proteins, all with no known homologues or function. As noted in the following examples, the SARS-CoV-2 RBM contains two anti-parallel beta strands (.beta.5 and .beta.6), a single disulfide bond and a 16-amino acid loop serving as the tacking segment that tacks the RBM to the core.

Thus, in certain embodiments, the native RBM may comprise residues 432 to 486 of the SARS-CoV-2 Spike protein. In more specific embodiments, the tacking segment may be an amino acid loop comprising residues 444 to 459 of the SARS-CoV-2 Spike protein. In some embodiments, the reconstituted RBM of the polypeptide of the disclosure may comprise at least one linker added to or replacing at least one of the tacking loop, any part or amino acid residue/s thereof or at least one RBM fragment or amino acid residue not directly involved with receptor and/or nAb/s interaction or binding. In some particular embodiments, the reconstituted RBM of the disclosure may comprise additional linker/s that may be located in other segments of the RBM. In some embodiments, the at least one linker of the disclosure may replace any amino acid sequence or amino acid residue/of the SARS-CoV-2 RBM that comprise at least part of the tacking loop as referred to herein.

It should be noted however, that in some embodiments, the reconstituted RBM of the disclosure may comprise at least one linker that replaces other fragments or amino acid residues of the native RBM, specifically, at least one linker that replaces fragments or amino acid residues not involved in the receptor-virus and/or nAb/s-virus interaction. These fragments or amino acid resides may be within or of out of the loop segment. Still further embodiments encompass reconstituted RBM polypeptides, wherein at least one amino acid residue/s or fragment/s of the RBM directly or indirectly involved in receptor and/or nAb/s binding is replaced with at least one linker.

In more specific embodiments the reconstituted RBM of the polypeptide of the disclosure may comprise an amino acid linker. In some embodiments, the linkers may comprise at least 1, 2, 3, 4, 5, 6, 7, 8, 9, 10 or more amino acid resides. In yet some more particular embodiments, such linker may comprise for example, 3, 4, 5, 6, 7, 8 or more amino acid residues.

In some specific embodiments, the polypeptide of the disclosure may comprise at least one reconstituted SARS-CoV-2 RBM comprising residues .sub.432STGNYNYKYRL.sub.443 as denoted by SEQ ID NO: 3 and .sub.460FSPDGKPCTPCTPPALNCYWPLNDYGFYTT.sub.486 as denoted by SEQ ID NO: 4 or any fragments, mutants or derivatives thereof, linked by or attached to said at least one linker. In some specific and non-limiting embodiments, at least one of the linker/s may replace the loop or any amino acid residue/s or any parts thereof, or any sequence comprising said loop or any parts or fragments thereof.

In yet some alternative embodiments, the polypeptide of the disclosure may comprise at least one reconstituted SARS-CoV-2 RBM that may be a mutated RBM having a K465I mutation. In more specific embodiments, such mutated reconstituted RBM may comprise residues .sub.432STGNYNYKYRL.sub.443 as denoted by SEQ ID NO: 3 and .sub.460FSPDGIPCTPCTPPALNCYWPLNDYGFYTT.sub.486 as denoted by SEQ ID NO:5 or any fragments or derivatives thereof, linked by said at least one linker. In some embodiments, said linker may replace the loop or any parts thereof, or any sequence comprising said loop or any parts thereof. In some particular and non-limiting examples, the reconstituted RBM of the disclosure may comprise .sub.432STGNYNYKYRL.sub.443 as denoted by SEQ ID NO: 3 and .sub.460FSPDGIPCTPCTPPALNCYWPLNDYGFYTT.sub.486 as denoted by SEQ ID NO: 5 or any fragments or derivatives thereof, linked by GEM. Such reconstituted RBM may be referred to by the disclosure as GEM465I.

Still further particular embodiments of the disclosure provide reconstituted RBM of the polypeptide of the disclosure that comprises .sub.432STGNYNYKYRL.sub.443 as denoted by SEQ ID NO: 3 and .sub.460FSPDGIPCTPCTPPALNCYWPLNDYGFYTT.sub.486 as denoted by SEQ ID NO: 5 or any fragments or derivatives thereof, linked by EEP. Such reconstituted RBM may be referred to by the disclosure as EEP465I.

The disclosure thus provides reconstituted RBMs, specifically, CoV-derived reconstituted RBMs polypeptides. These RBM polypeptides are in some embodiments, isolated and purified polypeptides. In some specific embodiments, these reconstituted RBM polypeptides may be recombinant polypeptides, that may in some embodiments recombinantly produced. However, the disclosure further encompasses reconstituted RBMs that are produced synthetically.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art.

The polypeptide of the disclosure are therefore considered as proteinaceous material. A “proteinaceous material” is any protein, or fragment thereof, or complex containing one or more proteins formed by any means, such as covalent peptide bonds, disulfide bonds, chemical crosslinks, etc., or non-covalent associations, such as hydrogen bonding, van der Waal's contacts, electrostatic salt bridges, etc.

The reconstituted RBM polypeptides of the disclosure are composed of an amino acid sequence. An ‘amino acid/s’ or an ‘amino acid residue/s’ can be a natural or non-natural amino acid residue/s linked by peptide bonds or bonds different from peptide bonds. The amino acid residues can be in D-configuration or L-configuration (referred to herein as D- or L-enantiomers). An amino acid residue comprises an amino terminal part (NH₂) and a carboxy terminal part (COOH) separated by a central part (R group) comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g. hydroxyproline, .gamma.-carboxyglutamate, and O-phosphoserine.

Thus, the reconstituted RBM polypeptides of the disclosure may comprise natural or non-natural amino acid residues, or any combination thereof.

Further, amino acids may be amino acid analogs or amino acid mimetics. Amino acid analogs refer to compounds that have the same fundamental chemical structure as naturally occurring amino acids, but modified R groups or modified peptide backbones, e.g. homoserine, norleucine, methionine sulfoxide, methionine methyl sulfonium. Amino acid mimetics refers to chemical compounds that have a structure that is different from the general chemical structure of an amino acid, but that function in a manner similar. Amino acids may be referred to herein by either their commonly known three letter symbols or by the one-letter symbols recommended by the IUPAC-IUB Biochemical Nomenclature Commission.

Further, the reconstituted RBM polypeptides of the disclosure may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions.

In the context of the present disclosure, within the meaning of the term ‘equivalent amino acid substitution’ as applied herein, is meant that in certain embodiments one amino acid may be substituted for another within the groups of amino acids indicated herein below: [0182] i) Amino acids having polar side chains (Asp, Glu, Lys, Arg, His, Asn, Gin, Ser, Thr, Tyr, and Cys); [0183] ii) Amino acids having non-polar side chains (Gly, Ala, Val, Leu, lie, Phe, Trp, Pro, and Met); [0184] iii) Amino acids having aliphatic side chains (Gly, Ala Val, Leu, ile); [0185] iv) Amino acids having cyclic side chains (Phe, Tyr, Trp, His, Pro); [0186] v) Amino acids having aromatic side chains (Phe, Tyr, Trp); [0187] vi) Amino acids having acidic side chains (Asp, Glu); [0188] vii) Amino acids having basic side chains (Lys, Arg, His); [0189] viii) Amino acids having amide side chains (Asn, Gln); [0190] ix) Amino acids having hydroxy side chains (Ser, Thr); [0191] x) Amino acids having sulphur-containing side chains (Cys, Met); [0192] xi) Neutral, weakly hydrophobic amino acids (Pro, Ala, Gly, Ser, Thr); [0193] xii) Hydrophilic, acidic amino acids (Gin, Asn, Glu, Asp), and [0194] xiii) Hydrophobic amino acids (Leu, Ile, Val).

Still further, the reconstituted RBM polypeptide of the disclosure of the disclosure may have secondary modifications, such as phosphorylation, acetylation, glycosylation, sulfhydryl bond formation, cleavage and the likes, as long as said modifications retain the functional properties of the original protein, specifically, the ability to interact with the viral receptor and the neutralizing antibodies. Secondary modifications are often referred to in terms of relative position to certain amino acid residues. For example, a certain sequence positioned carboxyl-terminal to a reference sequence within a polypeptide is located proximal to the carboxyl terminus of the reference sequence, but is not necessarily at the carboxyl terminus of the complete polypeptide.

The disclosure further encompasses any derivatives, enantiomers, analogues, variants or homologues of any of the reconstituted RBM polypeptides disclosed herein. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present disclosure. It should be noted that the reconstituted RBM polypeptides according to the disclosure can be produced either synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein, polypeptides that have deletions, substitutions, inversions or additions.

In some embodiments, derivatives refer to polypeptides, which differ from the polypeptides specifically defined in the present disclosure by insertions of amino acid residues. It should be appreciated that by the terms “insertions” or “deletions”, as used herein it is meant any addition or deletion, respectively, of amino acid residues to the polypeptides used by the disclosure, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the disclosure may occur in any position of the modified peptide, as well as in any of the N′ or C′ termini thereof. It should be appreciated that in cases the deletion/s or insertion/s are in the N or C-terminus of the peptide, such derivatives may be also referred to as fragments.

The reconstituted RBM polypeptide of the disclosure of the disclosure may all be positively charged, negatively charged or neutral. In addition, they may be in the form of a dimer, a multimer or in a constrained conformation, which can be attained by internal bridges, short-range cyclizations, extension or other chemical modifications.

The polypeptides of the disclosure can be coupled (conjugated) through any of their residues to another peptide or agent. For example, the polypeptides of the disclosure can be coupled through their N-terminus to a lauryl-cysteine (LC) residue and/or through their C-terminus to a cysteine (C) residue.

Further, the reconstituted RBM polypeptide of the disclosure may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the reconstituted RBM polypeptide may be extended at the N-terminus and/or C-terminus thereof with an N-acetyl group.

For every single peptide sequence defined by the disclosure and disclosed herein, this disclosure includes the corresponding retro-inverse sequence wherein the direction of the peptide chain has been inverted and wherein all or part of the amino acids belong to the D-series. It should be understood that the present disclosure includes embodiments wherein one or more of the L-amino acids is replaced with its D isomer.

In yet some further embodiments, the reconstituted RBM polypeptide of the disclosure of the disclosure may comprise at least one amino acid residue in the D-form. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations.

It should be appreciated that in some embodiments, the enantiomer or any derivatives of the reconstituted RBMs of the disclosure may exhibit at least one of enhanced activity, and superiority. In more specific embodiments, such derivatives and enantiomers may exhibit increased affinity to the nAbs or the viral receptor, enhanced stability, and increased resistance to proteolytic degradation.

The disclosure also encompasses any homologues of the polypeptides specifically defined by their amino acid sequence according to the disclosure. The term “homologues” is used to define amino acid sequences (polypeptide) which maintain a minimal homology to the amino acid sequences defined by the disclosure, e.g. preferably have at least about 65%, more preferably at least about 70%, at least about 75%, even more preferably at least about 80%, at least about 85%, most preferably at least about 90%, at least about 95% overall sequence homology with the amino acid sequence of any of the polypeptide as structurally defined above, e.g. of a specified sequence, and any derivatives, enantiomers and fusion proteins thereof.

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N-nor C-terminal extensions nor insertions or deletions shall be construed as reducing identity or homology. Methods and computer programs for the alignment are well known in the art.

In some embodiments, the present disclosure also encompasses polypeptides which are variants of, or analogues to, the polypeptides specifically defined in the disclosure by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art and disclosed herein before. Such conservatively modified variants are in addition to and do not exclude polymorphic variants, interspecies homologues, and alleles and analogous peptides of the disclosure.

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved.

As noted above, the peptides of the disclosure may be modified by omitting their N-terminal sequence. It should be appreciated that the disclosure further encompasses the omission of about 1, 2, 3, 4, 5, 6, 7, 8 and more amino acid residues from both, the N′ and/or the C′ termini of the peptides of the disclosure.

Certain commonly encountered amino acids which also provide useful substitutions include, but are not limited to, .beta.-alanine (.beta.-Ala) and other omega-amino acids such as 3-aminopropionic acid, 2,3-diaminopropionic acid (Dpr), 4-aminobutyric acid and so forth; .alpha.-aminoisobutyric acid (Aib); .epsilon.-aminohexanoic acid (Aha); .delta.-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (NIe); naphthylalanine (NaI); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,4-diaminobutyric acid (Dbu); 2,4-diaminobutyric acid (Dab); p-aminophenylalanine (Phe(pNH.sub.2)); N-methyl valine (MeVaI); homocysteine (hCys), homophenylalanine (hPhe) and homoserine (hSer); hydroxyproline (Hyp), homoproline (hPro), N-methylated amino acids (e.g., N-substituted glycine). Covalent Modifications of Amino Acids and the Peptide

Covalent modifications of the peptide are included and may be introduced by reacting targeted amino acid residues of the peptide with an organic derivatizing agent that is capable of reacting with selected side chains or terminal residues.

Cysteinyl residues most commonly are reacted with .alpha.-haloacetates (and corresponding amines) to give carboxymethyl or carboxyamidomethyl derivatives. Cysteinyl residues also are derivatized by reaction with bromotrifluoroacetone, .alpha.-bromo-.beta.-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylprocarbonate (pH 5.5-7.0) which agent is relatively specific for the histidyl side chain. Bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents reverses the charge of the lysinyl residues. Other suitable reagents for derivatizing .alpha.-amino-containing residues include imidoesters such as methylpicolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKa of the guanidine functional group. Furthermore, these reagents may react with the groups of lysine as well as the arginine .epsilon.-amino group.

Modification of tyrosyl residues has permits introduction of spectral labels into a peptide. This is accomplished by reaction with aromatic diazonium compounds or tetranitromethane. Most commonly. N-acetylimidizol and tetranitromethane are used to create O-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this disclosure.

Derivatization with bifunctional agents is useful for cross-linking the peptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3[(p-azidophenyl)dithio]propioimidate yield photoactivatable intermediates that are capable of forming crosslinks in the presence of light.

Other chemical modifications include hydroxylation of proline and lysine, phosphorylation of the hydroxyl groups of seryl or threonyl residues, methylation of the .alpha.-amino groups of lysine, arginine, and histidine side chains (Creighton, supra), acetylation of the N-terminal amine, and, in some instances, amidation of the C-terminal carboxyl.

Such chemically modified and derivatized moieties may improve the peptide's solubility, absorption, biological half-life, and the like. These changes may eliminate or attenuate undesirable side effects of the proteins in vivo. Adjuvants, Immune Stimulants and Immunogen Formulations

It should be appreciated that the disclosure further encompass any of the peptides of the disclosure referred herein, any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, and diethanolamine salts.

The present disclosure encompasses any fragment, derivative or analogue of any of the reconstituted RBM polypeptides of the disclosure. In certain embodiments, any of the polypeptides of the disclosure and derivatives thereof can bind the viral receptor as well as neutralizing antibodies (nAb/s), and more importantly, possess the ability to elicit the production of neutralizing antibodies in a subject vaccinated by the polypeptides of the disclosure. Thus, in some embodiments a “functional” reconstituted RBM polypeptide of the disclosure is a peptide possessing the ability to elicit the production of neutralizing antibodies, and/or inducing immunity against the infecting CoV/In certain embodiments, the reconstituted RBMs of the disclosure may be fused to additional peptide sequences. The disclosure further encompasses any fusion protein comprising the reconstituted RBMs of the disclosure as described herein. More specifically, additional peptide sequences can be added to the polypeptides (reconstituted RBMs) of the disclosure thereby forming fusion proteins, which act to promote stability, purification, and/or detection. For example, a reporter peptide portion (e.g., green fluorescent protein (GFP), .beta.-galactosidase, or a detectable domain thereof) can be used. Purification-facilitating peptide sequences include those derived or obtained from maltose binding protein (MBP), glutathione-S-transferase (GST), or thioredoxin (TRX).

Still further, in certain embodiments, the disclosure further encompasses any nucleic acid sequence encoding any of the reconstituted RBM polypeptides described herein, as well as any expression vector comprising said encoding nucleic acid sequence, or any host cell expressing the same. As used herein, the term ‘polynucleotide’ or a ‘nucleic acid sequence’ refers to a polymer of nucleic acids, such as deoxyribonucleic acid (DNA) or ribonucleic acid (RNA). As used herein. ‘nucleic acid’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

In this connection an “isolated polynucleotide” is a nucleic acid molecule that is separated from the genome of an organism. For example, a DNA molecule that encodes any of the reconstituted RBM polypeptides of the disclosure or any derivative, variant, fragment or fusion protein thereof that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species. The disclosure further relates to recombinant DNA constructs comprising the polynucleotides of the disclosure or variants, homologues or derivatives thereof. The constructs of the disclosure may further comprise additional elements such as promoters, regulatory and control elements, translation, expression and other signals, operably linked to the nucleic acid sequence of the disclosure. As used herein, the term “recombinant DNA” or “recombinant gene” refers to a nucleic acid comprising an open reading frame encoding one of the proteins of the disclosure.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Accordingly, the term control and regulatory elements includes promoters, terminators and other expression control elements. For instance, any of a wide variety of expression control sequences that control the expression of a DNA sequence when operatively linked to it may be used in these vectors to express DNA sequences encoding any desired protein using the method of this disclosure. A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein.

In yet a second aspect, the disclosure relates to a composition comprising an effective amount of at least one polypeptide comprising an amino acid sequence of at least one reconstituted RBM of a viral Spike protein, specifically, CoV Spike protein or any fragments thereof, any derivative, enantiomer, fusion protein, conjugate or polyvalent dendrimer thereof. More specifically, the reconstituted RBM of the disclosure may comprise at least one linker and at least one fragment of the native RBM. In some specific embodiments, the native RBM is a 30 to 200 amino acid sequence comprised within the RBD of said Spike protein forming a binding interface that interacts with the viral receptor. In more particular embodiments, the native RBM may be an extended 30 to 100 or more amino acid excursion, juxtaposed along the edge of the core of the RBD and tacked to the core via a tacking segment. More specifically, the reconstituted RBM may comprise at least one exogenous linker. In some embodiments, at least one of linker/s replace or may be added to at least one of: the tacking segment, any part or amino acid residue thereof and any RBM fragment or amino acid residue/s not directly involved in receptor and/or nAb/s binding, and RBM residues directly or indirectly involved in receptor and/or nAb/s binding. The composition of the disclosure may optionally further comprises at least one pharmaceutically acceptable carrier/s, excipient/s, auxiliaries, and/or diluent/s.

In certain embodiments, the composition of the disclosure may comprise a polypeptide comprising any of the reconstituted RBMs defined by the disclosure.

The term “pharmaceutical composition” in the context of the disclosure means that the composition is of a grade and purity suitable for prophylactic or therapeutic administration to human subjects and is present together with at least one of carrier/s, diluent/s, excipient/s and/or additive/s that are pharmaceutically acceptable. The pharmaceutical composition may be suitable for any mode of administration whether oral or parenteral, by injection or by topical administration by inhalation, intranasal spray or intraocular drops. More specifically, pulmonary, oral, transmucosal, intestinal or parenteral delivery, including intramuscular, subcutaneous and intramedullary injections as well as rectal, intrathecal, direct intraventricular, intravenous, intraocular injections or any other medically acceptable methods of administration may be considered as appropriate administration mode for the compositions of the disclosure.

Pharmaceutical compositions according to the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present disclosure also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

As noted above, any of the compositions of the disclosure may comprise pharmaceutically acceptable carriers, vehicles, adjuvants, excipients, or diluents. As used herein pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Each carrier should be both pharmaceutically and physiologically acceptable in the sense of being compatible with the other ingredients and not injurious to the subject. Formulations include those suitable for immersion, oral, parenteral (including subcutaneous, intramuscular, intravenous, intraperitoneal, implantation for slow release and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

In yet some further embodiments, the composition of the disclosure may be an immunogenic composition. Thus, in some embodiments, the immunogenic composition of the disclosure may induce an immune response in a subject. “Immune response” as used herein means the activation of a host's immune system, e.g., that of a mammal, in response to the introduction of antigen. The immune response can be in the form of a cellular or humoral response, or both.

### Vaccine and Immune Response

Still further aspect of the disclosure relates to an epitope based CoV vaccine comprising at least one polypeptide comprising an amino acid sequence of at least one reconstituted RBM of a viral Spike protein, specifically, CoV spike protein, more specifically, S protein, or of any fragment thereof, any derivative, enantiomer, fusion protein, conjugate or polyvalent dendrimer thereof. More specifically, the reconstituted RBM of the disclosure may comprise at least one linker and at least one fragment of the native RBM. In some specific embodiments, the native RBM is a 30 to 200 amino acid sequence comprised within the RBD of said Spike protein forming a binding interface that interacts with the viral receptor. The native RBM may be an extended 30 to 100 or more amino acid excursion, juxtaposed along the edge of the core of the RBD and tacked to the core via a tacking segment. In some specific embodiments the reconstituted RBM of the vaccine of the disclosure may comprise at least one exogenous linker. It should be noted that at least one of the linker/s may replace at least one of: the tacking segment, any part or amino acid residue thereof, any RBM fragment or amino acid residue/s not directly involved in receptor binding or any RBM fragment or amino acid residue directly or indirectly involved in said binding. In more specific embodiments, the vaccine of the disclosure optionally further comprises at least one pharmaceutically acceptable carrier/s, excipient/s, auxiliaries, and/or diluent/s.

In some embodiments, the vaccine provided by the disclosure may comprise at least one polypeptide comprising any of the reconstituted RBM as defined by the disclosure and described herein above. Provided herein are immunogenic compositions, such as vaccines, comprising at least one of reconstituted RBM polypeptide, specifically, reconstituted CoV RBMs, more specifically, reconstituted RBMs of SARS-CoV-2, any fragment, derivative, enantiomer, variant, conjugate and fusion protein thereof, or a combination thereof. The vaccine can be used to protect against any number of strains of CoVs, specifically, any strains or variants of SARS-CoV-2, thereby treating, preventing, and/or protecting against SARS-CoV-2 associated pathologies. The vaccine can significantly induce an immune response of a subject administered the vaccine, thereby protecting against and treating CoVs infections, specifically, SARS-CoV-2 infections.

Still further, when provided prophylactically, the reconstituted RBMs of the disclosure or any derivative, enantiomer, fusion protein or conjugate thereof, may be provided in advance of the CoV infection, specifically. SARS-CoV-2 infection, such as to patients or subjects who are at risk for being exposed to SARS-CoV-2 or who have been newly exposed to SARS-CoV-2, such as healthcare workers, blood products, or transplantation tissue, and other individuals who have been exposed to a body fluid that contains or may contain SARS-CoV-2. The prophylactic administration of the reconstituted RBMs of the disclosure or any derivative, enantiomer, fusion protein or conjugate thereof prevents, ameliorates, or delays SARS-CoV-2 infection. In subjects who have been newly exposed to SARS-CoV-2 but who have not yet displayed the presence of the virus (as measured by PCR or other assays for detecting the virus) in blood or other body fluid, efficacious treatment with the reconstituted RBMs of the disclosure or any derivative, enantiomer, fusion protein or conjugate thereof partially or completely inhibits or delays the appearance of the virus or minimizes the level of the virus in the blood or other body fluid of the exposed individual.

The efficacy of the reconstituted RBMs of the disclosure or any derivative, enantiomer, fusion protein or conjugate thereof, can be assessed in various ways well known to the skilled practitioner. For instance, one of ordinary skill in the art will understand that vaccine of the disclosure is efficacious in treating or inhibiting a SARS-CoV-2 infection in a subject by observing that the neutralizing antibodies induced thereby reduce viral load or delays or prevents a further increase in viral load. Viral loads can be measured by methods that are known in the art, for example, using PCR assays or antibody assays to detect the presence of SARS-CoV-2 nucleic acid to detect the presence of SARS-CoV-2 protein in a sample (e.g., blood or another body fluid) from a subject or patient, or by measuring the level of circulating anti-SARS-CoV-2 antibodies in the patient.

In some embodiments, the vaccine may induce a humoral immune response in the subject administered the vaccine or the immunogenic composition. In some embodiments, the induced humoral immune response may be specific for the specific CoV, specifically, SARS-CoV-2. The humoral immune response may be induced in the subject administered the vaccine by about 1.5-fold to about 100-fold, about 2-fold to about 90-fold, or about 3-fold to about 80-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more.

The humoral immune response induced by the vaccine may include an increased level of neutralizing antibodies associated with the subject administered the vaccine as compared to a subject not administered the vaccine. The neutralizing antibodies may be specific for the SARS-CoV-2, specifically, for the reconstituted RBMs of the disclosure. The neutralizing antibodies can provide protection against and/or treatment of SARS-CoV-2 infection and its associated pathologies in the subject administered the vaccine.

The humoral immune response induced by the vaccine may include an increased level of IgG antibodies associated with the subject administered the vaccine as compared to a subject not administered the vaccine.

The humoral response may be cross-reactive against two or more strains of the particular CoV, specifically, two or more strains of the SARS-CoV-2. The level of IgG antibody associated with the subject administered the vaccine may be increased by about 1.5-fold to about 100-fold, about 2-fold to about 50-fold, or about 3-fold to about 25-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more.

In yet some further embodiments, the vaccine can induce a cellular immune response in the vaccinated subject, such response may be specific for the reconstituted RBMs of the disclosure. The induced cellular immune response may include eliciting a CD8.sup.+ T cell response, that may according to certain embodiments, include the production of cytokines such as interferon-gamma (IFN-.gamma.), tumor necrosis factor alpha (TNF-alpha), interleukin-2 (IL-2), or any combinations thereof.

In yet some further embodiments, the cellular immune response induced by the vaccine can include eliciting a CD4.sup.+ T cell response. In some embodiments, the CD4.sup.+ T cells may produce IFN-.gamma. TNF-.alpha., IL-2, or a combination of IFN-.gamma. and TNF-.alpha.

The vaccine may further induce an immune response when administered to different tissues such as the muscle or skin. The vaccine can further induce an immune response when administered via electroporation, or injection, or subcutaneously, or intramuscularly.

Also provided herein are methods of treating, protecting against, and/or preventing disease in a subject in need thereof by administering the vaccine to the subject.

Administration of the vaccine to the subject may induce or elicit an immune response in the subject. The induced immune response can be used to treat, prevent, and/or protect against disease, for example, pathologies relating to CoV infections, specifically, SARS-CoV-2 infection. The induced immune response provided the subject administered the vaccine resistance to one or more SARS-CoV-2 strains.

The vaccine dose may range between 0.001 pg to 100 mg active ingredient, specifically, the reconstituted RBMs of the disclosure/kg body weight/time, and in some embodiments may be 0.01 μg to 100 mg reconstituted RBM/kg body weight/time. The vaccine may be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment may be 1, 2, 3, 4, 5, 6, 7, 8, 9, 10, or more.

The vaccine may be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a camel or any other camelids, such as the llama, alpaca, guanaco, and vicuna of South America, a horse, a cow, a pig, a sheep, a cat, a dog, a rat, or a mouse.

In yet some further embodiments, the subject may be an avian subject, specifically, wild or domestic birds. It should be noted that the vaccine can be administered prophylactically or therapeutically. In prophylactic administration, the vaccines may be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The reconstituted RBM polypeptide of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the reconstituted RBM polypeptide.

### Adjuvant

In some embodiments, the disclosed method and composition further includes an immunologic adjuvant to be co-administered with the recombinant polypeptide.

### Aluminum Salts

The clinically approved alum adjuvants consist of precipitates of aluminum phosphate and aluminum hydroxide to which antigens are adsorbed. Although traditionally thought to function primarily by forming a long-lasting depot for antigen and by promoting their uptake by antigen-presenting cells (APCs), it is now clear that innate immune stimulation plays a primary role in the adjuvant activity of alum (Lambrecht et al., 2009; Marrack et al., 2009). Alum is used primarily to enhance antibody production and does not utilize TLR for its function in vivo (Gavin et al., 2006). In humans, responses to proteins with alum tend to be a mix of Th2 and Th 1 cells (Didierlaurent et al., 2009); however, in mice alum induces a profoundly polarized Th2 cell response, with Th2 cell-dependent antibody isotypes, to nearly all protein antigens. Studies in vitro employing macrophages and DCs have demonstrated that, after lipopolysaccharide (LPS) priming, alum can activate the NLRP3 inflammasome to produce mature IL-1β (Li et al., 2007). This process appears to involve phagocytosis of alum crystals and lysosomal release of cathepsin B into the cytoplasm, where the enzyme localizes at the site of caspase-1-associated inflammasome activity (Hornung et al., 2008). Although the data supporting NLRP3 inflammasome triggering by alum in vitro are compelling, there is considerable controversy surrounding the role of this pathway in the adjuvant activity of alum in vivo (Lambrecht et al., 2009; Marrack et al., 2009). These conflicting findings may relate to the different alum-antigen formulations and/or immunization protocols used by the different laboratories involved. Whether the inflammasome-caspase-1-dependent processing of IL-1 and IL-18 plays a role in the strong Th2 cell polarization triggered by alum in mice is also not clear. Stimulation of the Th2 cell-promoting cytokines IL-4, IL-6, and IL-25 from innate cells by alum has been proposed as an alternative explanation for the strong Th2 cell polarization observed in mice (Lambrecht et al., 2009; Marrack et al., 2009; Serre et al., 2008).

In addition to inflammasome activation by alum itself, the adjuvant can also trigger necrotic cell death and the release of the endogenous danger signal uric acid. Indeed, injection of uricase has been shown to block the immunopotentiating effect of alum administered by the intraperitoneal route (Kool et al., 2008; Lambrecht et al., 2009). The current controversies concerning the mechanism of action of alum adjuvants underscore the need to determine which subset of the innate responses provoked by an adjuvant are specifically required for enhanced antibody or T cell responses.

### Oil-in-Water Emulsions

MF59 (Novartis) and AS03 (GlaxoSmithKline) are both oil-in-water emulsions based on squalene, an oil that is more readily metabolized than the paraffin oil used in Freund's adjuvants. MF59 is licensed in most of Europe for use with seasonal flu vaccines in the elderly, and both are used in approved pandemic flu vaccines. As a result, there are considerable human data comparing flu vaccination with these adjuvants to the same vaccine without adjuvant or with alum (Mbow et al., 2010). These emulsions stimulate stronger antibody responses, permit fewer doses and antigen dose sparing, and generate marked memory responses, with a mixed Th1-Th2 cell phenotype (Ott et al., 1995). MF59 induces substantial local stimulation, recruitment of DCs, granulocytes, and differentiation of monocytes into DCs (Seubert et al., 2008), as well as increased uptake of antigen by DCs (Dupuis et al., 1998). Intramuscular injection of MF59 leads to a pattern of induced genes that is both larger and distinct from that induced by either alum or a TLR9 agonist (Mosca et al., 2008).

### Saponin-Based Adjuvants, ISCOMs

Immunostimulatory complexes (ISCOMs) are cagelike nanoparticles composed of saponins purified from the bark of a South American tree, Quillaja saponaria, formulated with cholesterol, phospholipid, and antigen. Vaccine antigens need not be incorporated into the particles, and most current applications use a mixture of soluble antigens and the antigen-free particle, such as ISCOMATRIX. ISCOMs do not act through any identified PRR; however, they enhance antigen uptake and prolong retention by DCs in draining lymph nodes, induce activation of DCs, and lead to strong antibody and T cell responses (Maraskovsky et al., 2009). Although ISCOMs are potent enhancers of Th cells, they do not impose a bias to either a Th1 or Th2 cell response. Unlike most other adjuvants, ISCOMs enable substantial MHC class I presentation and induce both CD8+ and CD4+ T cell responses to a variety of soluble protein antigens in man (Davis et al., 2004) and experimental animals. ISCOMs appear to destabilize the endosomal membrane, allowing greater cytoplasmic access for codelivered antigens compared to other forms of antigen delivery (Schnurr et al., 2009). A heterogenous fraction of saponins, Quil A, is widely used for veterinary vaccines, and a highly purified species, QS-21, is currently being tested in human studies with several vaccine candidates.

### Adjuvants Targeting Pattern Recognition Receptors

In contrast to the complex and still incompletely understood adjuvants described above, an increasing focus has been to use natural ligands or synthetic agonists for well-defined PRRs as adjuvants, either alone or with various formulations. A number of these are now in clinical or late preclinical stages of development for multiple applications and have been the subject of research to clarify the basis of their adjuvant activity.

### TLR3 and RLR Ligands

The discovery that double-stranded viral RNA (dsRNA) is a potent activator of innate immunity was a seminal finding for understanding host immunity against viral infection (Alexopoulou et al., 2001). Synthetic analogs of dsRNA (i.e., Poly IC) have been used as adjuvants (Longhi et al., 2009; Stahl-Hennig et al., 2009; Trumpfheller et al., 2008) and can act through two distinct types of PRRs. Viral or synthetic dsRNA activates TLR3 in endosomes (Alexopoulou et al., 2001) or through cytosolic ribonucleic acid (RNA) helicases (RLR), such as retinoic acid-inducible gene-1 (RIG-I) and melanoma differentiation associated gene 5 (MDAS) (Kato et al., 2006). TLR3 mediates its effects through the adaptor TRIF (Alexopoulou et al., 2001), whereas RLR signal through the adaptor IFN-β promoter stimulatory-1 (Kato et al., 2006).

TLR3 activation in DCs induces IL-12 and type I IFN and improves MHC class II expression and cross-presentation (Davey et al., 2010; Jongbloed et al., 2010; Kadowaki et al., 2001; Lore et al., 2003; Poulin et al., 2010; Schulz et al., 2005; Wang et al., 2010). Stimulation of MDA-5, most notably from non-hematopoietic cells (Longhi et al., 2009; Wang et al., 2010), strongly enhances production of type I IFNs. Type I IFNs play a critical role in enhancing T and B cell immunity with dsRNA through a variety of mechanisms that include activation of DCs, NK cells, and direct effects on T cells (Blanco et al., 2001; Le Bon et al., 2006; Longhi et al., 2009). Several synthetic analogs of dsRNA (Poly IC, Poly ICLC, and Poly IC12U) have been used as adjuvants with soluble proteins, DC targeting constructs, or inactivated viral vaccines (Gowen et al., 2007; Stahl-Hennig et al., 2009; Trumpfheller et al., 2008). Poly IC activates both TLR3 and MDA, whereas Poly IU signals through TLR3 only. Activation of both TLR3 and MDAS optimizes the magnitude and durability of Th 1 cell immunity and CD8+ T cell immunity compared to either pathway alone. This highlights a central feature of the potency of Poly IC by inducing TLR3 activation of DCs directly and type I IFNs through MDA-5 (Longhi et al., 2009).

The formulation of Poly IC has a critical influence on its potency. Thus, long dsRNA is required to activate MDA-5 (Kato et al., 2008). Furthermore, complexing Poly IC with poly-L-lysine and carboxymethylcellose (poly ICLC) prolongs the adjuvant effect in vivo (Levy et al., 1975; Stahl-Hennig et al., 2009). Collectively, an optimally formulated Poly IC is an effective adjuvant for inducing broad-based adaptive immunity through both TLR and RLR signaling pathways.

### TLR4 Ligands

Bacterial lipopolysaccharides have long been recognized as potent adjuvants, but their pyrogenic activity has precluded use as an adjuvant in man. Pioneering work from Ribi (Qureshi et al., 1982) led to the development of less toxic preparations of LPS, and ultimately to the substantially detoxified derivative monophosphoryl lipid A (MPL). MPL, principally formulated with antigens and alum, is now a component of licensed vaccines for HBV and papilloma and has proven to be both safe and effective (Casella and Mitchell, 2008). Both LPS and MPL are recognized specifically by TLR4, but MPL leads to signaling only through the TRIF adaptor, whereas LPS leads to TLR4 activation through both the TRIF and MyD88 pathways (Mata-Haro et al., 2007), the latter pathway resulting in high levels of many inflammatory cytokines, prominently TNF-α. MPL formulated on alum (AS04) stimulates a polarized Th 1 cell response in contrast to the mixed Th1-Th2 cell response of alum alone (Casella and Mitchell, 2008; Didierlaurent et al., 2009). Much of the adjuvant activity of this mixture can be attributed to the MPL component, although alum helps prolong stimulation by MPL (Didierlaurent et al., 2009).

### TLR5 Ligands

Bacterial flagellin has long been known to be a potent T cell-independent antigen, but the finding that flagellin from many species was a ligand for TLR5 suggested its potential as an adjuvant. Although flagellin itself can be an adjuvant when mixed with antigens, current application is primarily by generation of fusion proteins of recombinant vaccine antigens and flagellin (Huleatt et al., 2007). Unlike many other TLR agonists, flagellin tends to produce mixed Th 1 and Th2 cell responses rather than strongly polarized Th 1 cell patterns (Huleatt et al., 2007). Antibody production to fusion proteins requires TLR5 expression (McDonald et al., 2007), but optimum adjuvant effect in mice requires expression of the TLR signaling adaptor MyD88 in both hematopoietic and nonhematopoeitic (radioresistant) cell types (Sanders et al., 2008). Bacterial flagellins can also signal through inflammasomes that contain Nlrc4 (also known as IPAF) (Miao and Warren, 2010), although it is not known whether this pathway contributes to the adjuvant activity of flagellin.

### TLR7 and TLR8 Ligands

Guanosine- and uridine-rich ssRNA were first identified as natural agonists for TLR7 and 8 (Diebold et al., 2004; Heil et al., 2004; Lund et al., 2004). Because ssRNA is rapidly degraded by extracellular RNases, using it as an adjuvant without substantial modification or formulation is unpromising. However, a number of small synthetic compounds originally developed as type I IFN inducers, including imidazoquinolines (Imiquimod, TLR7 and Resiquimod, TLR7-TLR8) and guanosine and adenosine analogs, have been shown to activate TLR7, TLR8, or both (Gorden et al., 2005; Heil et al., 2003; Hemmi et al., 2002). TLR7 and TLR8 are expressed in endosomes, but not on the cell surface, and both mediate their effects through MyD88-dependent signaling (Hemmi et al., 2002).

Important differences exist between mice and humans with regard to tissue expression and function of TLR7 and TLR8. In both species, TLR7 is expressed in B cells, neutrophils, and plasmacytoid DCs (pDCs); however, in mice TLR7 is expressed by macrophages and CD8−, but not CD8+, DC subsets (Iwasaki and Medzhitov, 2004). TLR8, in contrast, is expressed by monocyte lineage cells and myeloid DCs in man, whereas it may not be a functional receptor in mice (Jurk et al., 2002). Activation of TLR7 and TLR8 in human pDCs and mDCs, respectively, increases the expression of costimulatory molecules and production of type I IFN and IL-12 (Jarrossay et al., 2001; Kadowaki et al., 2001; Lore et al., 2003). A bispecific TLR7-TLR8 agonist may be more effective than a monospecific agonist by activating multiple DC subsets and B cells to induce cytokines optimal for Th1 cell immunity, cross-presentation, and antibody production. Small TLR7 or 8 agonists are not very effective as adjuvants when simply mixed with antigens, but can be substantially improved by formulation with or conjugation to the antigen (Wille-Reece et al., 2005, 2006; Wu et al., 2007).

### TLR9—CpG-ODN and Formulated DNA

TLR9 is the only endosomal PRR specific for DNA and mediates a potent innate response to bacterial and viral DNA (Blasius and Beutler, 2010). Sequence motifs containing the CpG dinucleotide are preferentially recognized; however, specific base sequences only partly account for TLR9 binding. The sugar-phosphate backbone is also integral to recognition by TLR9 (Haas et al., 2008). Synthetic 18-25 base oligodeoxynucleotides (ODN) with optimized CpG motifs (CpG-ODN) have been studied extensively as adjuvants, either soluble or formulated in nanoparticles (Marshall et al., 2004) or virus-like particles (Jennings and Bachmann, 2009). CpG-ODN enhance antibody responses and strongly polarize Th cell responses to Th1 and away from Th2 cell responses (Kobayashi et al., 1999; Tighe et al., 2000). TLR9 has a relatively restricted cellular distribution, especially in man, with the two major APC types being B cells and pDCs (Campbell et al., 2009). Studies with a DC-specific deletion of TLR signaling in mice indicate that DC recognition is much more important for the antibody-enhancing activity of CpG-ODN than B cell expression (Hou et al., 2008). However, in primates, myeloid DCs, thought to be the principal antigen-presenting DCs, are TLR9 negative, suggesting either that activated PDC are sufficient for the adjuvant effect of CpG-ODN or that myeloid DCs become activated in the lymph node by indirect means (Teleshova et al., 2006).

### Codelivery of Antigens and PRR Ligands

The immune system is optimized to generate adaptive responses to microbial antigens delivered to APCs in intimate association with PRR ligands, as would be the case for viral and microbial infections and live attenuated vaccines. For subunit vaccine candidates, codelivery has been accomplished by covalent coupling of TLR7-TLR8 (Wille-Reece et al., 2005; Wu et al., 2007) and TLR9 (Tighe et al., 2000) to purified proteins or by constructing recombinant fusion proteins consisting of antigen and the TLR5 ligand flagellin (Huleatt et al., 2007). In these examples, the potency of the linked vaccine is 10-100 times that of a comparable mix of separate components. In the case of CpG-ODN conjugates, coupling of an ODN enhances antigen uptake and cross-presentation in DCs, although the enhanced uptake is not TLR9 dependent (Heit et al., 2003). Co-delivery of antigens and PRR ligands can also be accomplished by association—covalent or noncovalent—of both within a larger particulate structure. Examples include virus-like particles (Jennings and Bachmann, 2009) and synthetic nano- and micro-particles (O'Hagan and De Gregorio, 2009).

The immune system is optimized to generate adaptive responses to microbial antigens delivered to APCs in intimate association with PRR ligands, as would be the case for viral and microbial infections and live attenuated vaccines. For subunit vaccine candidates, codelivery has been accomplished by covalent coupling of TLR7-TLR8 (Wille-Reece et al., 2005; Wu et al., 2007) and TLR9 (Tighe et al., 2000) to purified proteins or by constructing recombinant fusion proteins consisting of antigen and the TLR5 ligand flagellin (Huleatt et al., 2007). In these examples, the potency of the linked vaccine is 10-100 times that of a comparable mix of separate components. In the case of CpG-ODN conjugates, coupling of an ODN enhances antigen uptake and cross-presentation in DCs, although the enhanced uptake is not TLR9 dependent (Heit et al., 2003). Co-delivery of antigens and PRR ligands can also be accomplished by association—covalent or noncovalent—of both within a larger particulate structure. Examples include virus-like particles (Jennings and Bachmann, 2009) and synthetic nano- and micro-particles (O'Hagan and De Gregorio, 2009).

The enhanced efficiency of this codelivery may be simply quantitative—uptake of enough linked antigen for effective presentation will inherently provide a stimulatory amount of the linked PRR ligand, and enhanced uptake would lead to preferential presentation of the linked antigen. However, codelivery may also lead to preferential handling of antigens associated with PRR ligands, by facilitating antigen presentation at the level of individual lysosomes (Iwasaki and Medzhitov, 2010). A number of vaccine candidates with this strategy have reached early stage clinical studies, and this represents one of the most promising new directions in vaccine development.

### Adjuvant Combinations

One important lesson from studies of live attenuated vaccines is that activation of multiple innate receptors may be more effective than activation of a single pathway (Querec et al., 2006). This is logical, because redundant pathways of innate responsiveness would increase the likelihood of dealing successfully with an infection via a limited number of PRRs. Studies in vitro with defined combinations of TLR ligands support this idea (Trinchieri and Sher, 2007) and suggest combinations that may be especially useful for adjuvants. The very effective adjuvant systems developed by GlaxoSmithKline take this approach, combining MPL and alum (AS04) or MPL, QS-21, and either oil-in-water emulsion (AS02) or liposomes (AS01), and many more combinations are in late preclinical or early clinical stages of development.

### Formulation and Delivery

The vaccine can be delivered via a variety of routes. Typical delivery routes include parenteral administration, e.g., intradermal, intramuscular or subcutaneous delivery. Other routes include oral administration, intranasal, intravaginal and mucosal administration (such as intranasal, oral, intratracheal, and ocular).

The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant.

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices. Liposomes can consist of phospholipids or other lipids, and can be nontoxic, physiologically acceptable and metabolizable carriers that are relatively simple to make and administer.

Vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the disclosure methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof. Mucosal vaccines may be, for example, liquid dosage forms, such as pharmaceutically acceptable emulsions, solutions, suspensions, syrups, and elixirs. Excipients suitable for such vaccines include, for example, inert diluents commonly used in the art, such as, water, saline, dextrose, glycerol, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. Excipients also can comprise various wetting, emulsifying, suspending, flavoring (e.g., sweetening), and/or perfuming agents.

Oral mucosal vaccines also may, for example, be tableted or encapsulated for convenient administration. Such capsules or tablets can contain a controlled-release formulation. In the case of capsules, tablets, and pills, the dosage forms also can comprise buffering agents, such as sodium citrate, or magnesium or calcium carbonate or bicarbonate.

Tablets and pills additionally can be prepared with enteric coatings.

It is contemplated that the vaccine may be administered via the human, camel or avian patient's drinking water and/or food.

“Parenteral administration” that is also contemplated by the disclosure includes subcutaneous injections, submucosal injections, intravenous injections, intramuscular injections, intrasternal injections, transcutaneous injections, and infusion. Injectable preparations (e.g., sterile injectable aqueous or oleaginous suspensions) can be formulated according to the known art using suitable excipients, such as vehicles, solvents, dispersing, wetting agents, emulsifying agents, and/or suspending agents. These typically include, for example, water, saline, dextrose, glycerol, ethanol, corn oil, cottonseed oil, peanut oil, sesame oil, benzyl alcohol, benzyl alcohol, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), propylene glycol, and/or polyethylene glycols. Excipients also may include small amounts of other auxiliary substances, such as pH buffering agents.

The vaccine may include one or more excipients that enhance the vaccinated patient's immune response (which may include an antibody response, cellular response, or both), thereby increasing the effectiveness of the vaccine. The adjuvant(s) may be a substance that has a direct (e.g., cytokine or Bacille Calmette-Guerin (“BCG”)) or indirect effect (liposomes) on cells of the canine patient's immune system. Examples of often suitable adjuvants include oils (e.g., mineral oils), metallic salts (e.g., aluminum hydroxide or aluminum phosphate), bacterial components (e.g., bacterial liposaccharides, Freund's adjuvants, and/or MDP), plant components (e.g., Quil A), and/or one or more substances that have a carrier effect (e.g., bentonite, latex particles, liposomes, and/or Quil A, ISCOM). As noted above, adjuvants also include, for example, CARBIGEN™ and carbopol. It should be recognized that this disclosure encompasses both vaccines that comprise an adjuvant(s), as well as vaccines that do not comprise any adjuvant.

It is contemplated that the vaccine may be freeze-dried (or otherwise reduced in liquid volume) for storage, and then reconstituted in a liquid before or at the time of administration. Such reconstitution may be achieved using, for example, vaccine-grade water.

### Disease Prevention

It is another purpose of the present disclosure to combat CoVs infections in animals, particularly those relevant for infections in humans. Those relevant to SARS-CoV-2 include monkeys. Himalayan palm civets, raccoon dogs, cats, dogs, and rodents. Further, CoVs also cause a range of diseases in farm animals and domesticated pets, some of which can be serious and are a threat to the farming industry. Economically significant CoVs of farm animals include porcine CoV (transmissible gastroenteritis CoV, TGE) and bovine CoV, which both result in diarrhea in young animals. Feline CoVs include two forms.

Spontaneous mutations in feline enteric CoVs can result in feline infectious peritonitis (FIP)—a disease associated with high mortality. Similarly, there are two types of CoVs that infect ferrets. Ferret enteric CoV causes a gastrointestinal syndrome known as epizootic catarrhal enteritis (ECE), and a more lethal systemic version of the virus (like FIP in cats) known in ferrets as ferret systemic coronavirus (FSC). There are two types of canine CoV (CCoVs), one that causes mild gastrointestinal disease and one that has been found to cause respiratory disease. Mouse hepatitis virus (MHV) is a coronavirus that causes an epidemic murine illness with high mortality, especially among colonies of laboratory mice. In chickens, the infectious bronchitis virus (IBV), a CoV, targets not only the respiratory tract but also the uro-genital tract, and can spread to other organs. The vaccine of the disclosure as well as any of the compositions and methods described herein may be applicable for any of the disorders mentioned herein.

As noted herein before, the inventive concept behind the present disclosure is based on deep understanding of the unique structural/functional properties of the viral S1 protein and its components. This term herein refers to one of the functional subunits of the CoV Spike glycoprotein, a class I viral fusion protein that forms the characteristic spikes, or peplomers, found on the viral surface that mediate virus attachment, fusion, and entry into the host cell, and thereby determines the tropism of the virus. During virus maturation, Spike glycoprotein is cleaved into two subunits: S1, which binds to receptors in the host cell, and S2, which mediates membrane fusion.

In other words, herein this term refers to a CoV protein referred to by terms Coronavirus Spike Glycoprotein, Spike Protein, Spike Glycoproteins S1. Spike Gp S1, or term under MeSH Unique ID: D064370.

In some embodiments, this term refers to the SARS-CoV-2 S1 glycoprotein referred to by SARS coronavirus ShanghaiQXCI S1. GenBank Protein Accession: AAR86788.1, accession AY463059.1.

Different putative S1 sequences from SARS-CoV-2 isolates can be obtained from NCBI. It should be therefore appreciated that in certain embodiments, each of these S1 proteins is encompassed by the disclosure.

Table 1 lists GenBank accession numbers for 102 S gene sequences of SARS-CoV-2s

TABLE-US-00001 TABLE 1 Epidemic phases Strains Accession Epidemic Strains Accession 02-04 02-03 SZ1 AY304489 03-late GZ-B AY394978 SZ3 AY304486 GZ-C AY394979 interspecies phase SZ13 AY304487 epidemic TOR2 AY274119 SZ16 AY304488 group URBANI AY278741 epidemic WHU AY394850 group 03-04 PC4-13 AY613948 (56 sequences) HKU-39849 AY278491 PC4-115 AY627044 CUHK-SU10 AY282752 phase PC4-127 AY613951 CUHK-LC2 AY394999 (17 sequences) PC4-136 AY613949 CUHK-LC4 AY395001 PC4-137 AY627045 FRANKFURT AY291315 PC4-145 AY627046 TW3 AY502926 PC4-199 AY627047 TW8 AY502931 PC4-205 AY613952 TW10 AY502923 PC4-227 AY613950 TW11 AY502924 PC4-241 AY627048 TWH AP006557 GD03T13 AY525636 TWK AP006559 GZ03-01 AY568539 TWS AP006560 GZ03-02 AY613947 Sinol-11 AY485277 03-early-mid epidemic GZO2 AY390556 Sino3-11 AY485278 group HGZ8L1-A AY394981 Sin845 AY559093 (27 sequences) ZS-A AY394997 Sin849 AY559086 ZS-B AY394996 Sin850 AY559096 ZS-C AY395003 Sin852 AY559082 HSZ-Bb AY394985 SIN2677 AY283795 HSZ-Bc AY394994 SIN2748 AY283797 HSZ-Cb AY394986 Sin3765V AY559084 HSZ-Cc AY394995 TC1 AY338174 HGZ8L1-B AY394982 TC2 AY338175 GZ50 AY304495 TC3 AY348314 GZ-A AY394977 AS AY427439 JMD AY394988 A11S AY345986 BJ01 AY278488 A7N AY345987 BJ02 AY278487 STL2 AY345988 BJ03 AY278490 TW1 AY291451 BJ04 AY279354 TW2 AY502925 CUHK-W1 AY278554 TW4 AY502927 HZS2-A AY394983 TW5 AY502928 HZS2-Bb AY395004 TW6 AY502929 HZS2-CAY394992 TW7 AY502930 HZS2-D AY394989 TW9 AY502932 HZS2-E AY394990 TWC AY321118 HZS2-Fb AY394987 TWJ AP006558 HZS2-Fc AY394991 TWY AO996561 HCZ8L-2 AY394993 GD69 AY313906 NS-1 AY508724 HSR AY323977 Sin847AY559095 Sin848 AY559085 Outgroup B24 DQ022305 SIN2774 AY283798 (2 sequences) B41 DQ084199 SIN2500 AY283794 PUMC01 AY350750 PUMC02 AY357075 PUMC03 AY357076 SIN2679 AY283796 CUHK-LC1 AY394998 CUHK-LC3 AY395000 CUHK-LC5 AY395002

In yet some other embodiments, the term S1 protein refers to the MERS CoV S1 glycoprotein referred to by GenBank: AHX00731.1 GI:612348173. Locus AHX00731. In this connection, the disclosure further refers to the RBD as denoted by residues 381-588 or residues 367-606 of the MERS CoV S1.

Further, the present disclosure pertains to a specific region within the Spike Gp S1, specifically the Receptor Binding Domain (RBD), and more specifically the Receptor Binding Motif (RBM), as being responsible for the S1 bi-functional properties, namely host receptor binding and host immunity receptors. In the case of SARS-CoV-2, it has been demonstrated that the infection is mediated through the interaction of the viral Spike protein (1255 amino acids) and its cellular receptor Angiotensin-Converting Enzyme 2 (ACE2). The SARS-CoV-2 RBD harbors an extended excursion that contacts ACE2, which is RBM. It has been further demonstrated that RBM is a major antigenic determinant able to elicit production of neutralizing antibodies. Hence, the role of the RBM is a bi-functional bioactive surface that can be demonstrated by antibodies such as the neutralizing human anti-SARS monoclonal antibody (mAb) 80R which targets the RBM and competes with the ACE2 receptor for binding.

A further aspect of the disclosure provides a method for preventing, inhibiting, reducing, eliminating, protecting or delaying the onset of an infection or an infectious clinical condition caused by coronavirus in a subject in need thereof. More specifically, the method comprising the step of administrating to the subject an effective amount of at least one polypeptide comprising an amino acid sequence of at least one reconstituted RBM of CoV Spike protein, or of any fragment thereof, any derivative, enantiomer, fusion protein, conjugate or polyvalent dendrimer thereof or of any composition or vaccine comprising the same. In some embodiments, the reconstituted RBM of the disclosure may comprise at least one linker and at least one fragment of the native RBM. In further specific embodiments, the native RBM is a to 200 amino acid sequence comprised within the RBD of said Spike protein forming a binding interface that interacts with the viral receptor. In certain specific embodiments, the RBM is an extended 30 to 100 or more amino acid excursion, juxtaposed along the edge of the core of the RBD and tacked to the core via a tacking segment. In certain embodiments, the reconstituted RBM comprises at least one exogenous linker. It should be noted that in certain embodiments, at least one of said linker/s replaces at least one of the tacking segment, any part or amino residue/s thereof, any RBM fragment or amino acid residue/s not directly involved in receptor and/or nAb/s binding and any RBM fragment or amino acid residue/s directly or indirectly involved in receptor and/or nAb/s binding.

In more specific embodiments, any of the polypeptides of the disclosure that comprise any of the reconstituted RBM polypeptides defined by the disclosure, or any compositions or vaccines thereof, may be used by the method of the disclosure.

It should be noted that the disclosure further encompasses methods for treating and preventing infectious disease caused by viral pathogens, specifically, CoVs such as SARS-CoV-2. More specifically, SARS-CoV-2 associated disorders include but are not limited to respiratory illness (in particular severe acute respiratory illness) with symptoms of fever, cough, and/or shortness of breath. Pneumonia is a common finding on examination. Severe disease from SARS-CoV-2 infection can cause respiratory failure that requires mechanical ventilation and support in an intensive-care unit. Gastrointestinal symptoms, including diarrhea, nausea/vomiting have also been reported. Symptoms also include organ failure, especially of the kidneys, or septic shock. The virus appears to cause more severe disease in older people, in people with weakened immune systems, and in people with background illnesses or chronic diseases as for example diabetes, cancer and chronic lung disease. SARS-CoV-2 associated disorders or conditions include high fever (temperature greater than 100.4.degree. F. or 38.0.degree. C.), headache, mild respiratory symptoms, diarrhea, dry cough and develop pneumonia. The term “treatment” in accordance with disorders associated with infectious conditions may refer to one or more of the following: elimination, reducing or decreasing the intensity or frequency of disorders associated with said infectious condition. The treatment may be undertaken when disorders associated with said infection, incidence is beginning or may be a continuous administration, for example by administration every 1 to 14 days, to prevent or decrease occurrence of infectious condition in an individual prone to said condition. Such individual may be for example a subject having a compromised immune-system, in case of cancer patients undergoing chemotherapy or HIV infected subjects. Thus, the term “treatment” is also meant as prophylactic or ameliorating treatment.

The term “prophylaxis” refers to prevention or reduction the risk of occurrence of the biological or medical event, specifically, the occurrence or re occurrence of disorders associated with infectious disease, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the disclosure are particularly effective in the prophylaxis, i.e., prevention of conditions associated with infectious viral disease. Thus, subjects administered with said compositions are less likely to experience symptoms associated with said infectious condition that are also less likely to re-occur in a subject who has already experienced them in the past.

The term “amelioration” as referred to herein, relates to a decrease in the symptoms, and improvement in a subject's condition brought about by the compositions and methods according to the disclosure, wherein said improvement may be manifested in the forms of inhibition of pathologic processes associated with any infectious viral disease, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the disclosure described herein.

The terms “delay”, “delaying the onset”, “retard” and all variations thereof are intended to encompass the slowing of the progress and/or exacerbation of an infectious disease, specifically, of CoV infection and their symptoms slowing their progress, further exacerbation or development, so as to appear later than in the absence of the treatment according to the disclosure.

As used herein, “disease”, “disorder”, “condition” and the like, as they relate to a subject's health, are used interchangeably and have meanings ascribed to each and all of such terms.

The present disclosure relates to the treatment of subjects, or patients, in need thereof. By “patient” or “subject in need” it is meant any organism who may be affected by the above-mentioned conditions, and to whom the vaccinating and treatment methods herein described is desired, including humans, domestic and non-domestic mammals such as canine and feline subjects, bovine, simian, equine and murine subjects, rodents, domestic or wild birds, aquaculture, fish and exotic aquarium fish. It should be appreciated that the treated subject may be also any reptile or zoo animal. More specifically, the composition/s and method/s of the disclosure are intended for mammals or avian subjects. By “mammalian subject” is meant any mammal for which the proposed therapy is desired, including human, camelids, bats, equine, canine, and feline subjects, most specifically humans. It should be noted that specifically in cases of non-human subjects, the method of the disclosure may be performed using administration via injection, drinking water, feed, spraying, oral gavage and directly into the digestive tract of subjects in need thereof.

Single or multiple administrations of the compositions or vaccines of the disclosure are administered depending on the dosage and frequency as required and tolerated by the patient. In any event, the composition should provide a sufficient quantity of the reconstituted RBMs of the disclosure to effectively vaccinate and thereby treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Still further, the reconstituted RBMs of the disclosure or any compositions or kits thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month.

The application of the reconstituted RBMs of the disclosure or any compositions or kits thereof may last up to a day, two days, three days, four days, five days, six days, a week, two weeks, three weeks, four weeks, a month, two months three months or even more. Specifically, application may last from one day to one month. Most specifically, application may last from one day to 7 days.

The disclosure thus provides methods for inhibiting and preventing viral infection, specifically. CoV infections in a subject. It should be appreciated that such method may results in an inhibition, reduction, elimination, attenuation, retardation, decline, prevention or decrease of at least about 5%-99.9999%, about 10%-90%, about 15%-85%, about 20%-80%, about 25%-75%, about 30%-70%, about 35%-65%, about 40%-60% or about 45%-55%, and more specifically may be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the CoV infections, or infectious condition associated therewith as discussed above.

Still further, the disclosure provides a method of inducing an immune response against a viral infection, specifically, against coronavirus in a subject in need thereof. The method comprising administering to the subject an immunogenic effective amount of at least one polypeptide comprising an amino acid sequence of at least one reconstituted RBM of CoV Spike protein or any fragments thereof, any derivative, enantiomer, fusion protein, conjugate or polyvalent dendrimer thereof, or of any composition or vaccine comprising the same. More specifically, the native RBM is comprised within the Receptor Binding Domain (RBD) of said Spike protein forming a binding interface that interacts with the viral receptor. The RBM is an extended 30 to 100 or more amino acid excursion, juxtaposed along the edge of the core of the RBD and tacked to the core via a tacking segment. More specifically, the reconstituted RBM comprises at least one exogenous linker. In some embodiments, at least one of said linker/s may replace or added to at least one of: the tacking segment, any part or amino acid residue/s thereof and any RBM fragment or amino acid residue/s not directly involved in receptor binding.

It should be appreciated that any of the reconstituted RBM defined by the disclosure as well as any polypeptides, compositions or vaccines comprising these reconstituted RBMs, may be used for any of the methods of the disclosure. In yet some further embodiments any of the SARS-CoV-2 polypeptides of the disclosure, specifically, and of the polypeptides comprising the amino acid residues 319-545 of the SARS-CoV-2 spike protein, may be used in the methods of the disclosure.

Still further embodiments relate to the methods of the disclosure that may be applicable for any Coronavirus infection or any related disorders. Such infection may be caused by any CoV infecting mammalian subject/s, avian subject/s or any other vertebrates.

In more specific embodiments, the methods of the disclosure may be applicable for infections caused by coronavirus such as SARS-CoV-2.

The disclosure further provides the use of the polypeptide as defined by the disclosure in the preparation of an epitope based CoV vaccine for inducing an immune response against a coronavirus in a subject in need thereof.

It is conceived that the above described approach is applicable to any viral pathogen, specifically, any CoV that has the same general layout/structure with a RBD juxtaposed to a core. For any specific case, the inventors propose to remove/delete the “loop” that functions for RBM tacking to the body of RBD core, any parts thereof or any RBM fragment or amino acid residue not involved, participating or essential to receptor interaction and binding, and thus faces in the opposite direction relative to the binding surface that interacts with the receptor and is the target of neutralizing antibodies. As a result of this deletion, a gap is generated that requires a linker. The inventors have presently demonstrated how to design of a potential linker, in terms of length and composition, by introducing the concept of combinatorial linkers and conformer libraries, and a method for screening these libraries with concrete reagents that have affinity for RBM, i.e. the receptor and neutralizing antibodies.

More specifically, the selection of the correct linker length and composition is based on a pull down and affinity selection of conformers bearing the correct linker, and thereby reconstitution of a recognizable conformation. In the example of SARS, the gap was about 7A and ultimately was bridged with linker 3 and 4 residues in length. With regard to MERS, the same concept is applied. Composition of the successful linkers indicates that they are not random, but rather represent a motif in themselves, thus illustrating the uniqueness of these linkers and the fact that affinity selection not only works but apparently is necessary to discriminate the correct linkers from the vast collection of all other linkers in a conformer library.

The immunogenic peptides conceived by the present disclosure are applicable for generation of specific vaccines and vaccine-based therapeutic compositions targeting CoVs. A number of methods for producing specific formulation for vaccines are known in the art. A rational systematic approach for the development of vaccine formulations was provided for example by Morefield G. L. in AAPS J. 2011. Vol. 13(2). pp. 191-200.

Thus, in a further aspect, the disclosure further provides a method for producing epitope based CoV vaccine or immunogenic composition comprising reconstituted RBM. More specifically, the method comprising the step of:
(a) preparing reconstituted functional RBM/s of a CoV Spike protein, specifically, by the method as defined by the disclosure; and (b) admixing at least one of said reconstituted functional RBM/s of a CoV Spike protein or any derivative or enantiomer thereof, or any fusion protein, conjugate, or polyvalent dendrimer comprising the same with at least one adjuvant/s, carrier/s, excipient/s, auxiliaries, and/or diluent/s.

In yet some further embodiments, the reconstituted RBMs of the disclosure or any derivative, enantiomer, fusion protein or conjugate thereof may be presented in the vaccines of the disclosure as a polyvalent antigen by incorporation thereof in a polyvalent dendrimer. This embodiment is based on the knowledge in the art that a multiple antigen peptide carrying a multiplicity of epitopes induces superior immune responses compared to responses following immunization with corresponding equal amounts of monovalent epitopes.

Thus, in some embodiments, the present disclosure is intended to broadly encompass antigenic products carrying multiple copies of the reconstituted RBM polypeptides of the present disclosure an in a multiple antigen peptide system.

The present dendritic polymers are antigenic products in which the reconstituted RBM polypeptides are covalently bound to the branches that radiate from a core molecule. These dendritic polymers are characterized by higher concentrations of functional groups per unit of molecular volume than ordinary polymers. Generally, they are based upon two or more identical branches originating from a core molecule having at least two functional groups. The polymers are often referred to as dendritic polymers because their structure may be symbolized as a tree with a core trunk and several branches. Unlike a tree, however, the branches in dendritic polymers are substantially identical.

The dendrite system has been termed the “multiple antigen peptide system” (MAPS), which is the commonly used name for a combination antigen/antigen carrier that is composed of two or more, usually identical, antigenic molecules, specifically, the reconstituted RBM polypeptides of the disclosure covalently attached to a dendritic core which is composed of principal units which are at least bifunctional/difunctional. Each bifunctional unit in a branch provides a base for added growth. The dendritic core of a multiple antigen peptide system may be composed of lysine molecules. For example, a lysine is attached via peptide bonds through each of its amino groups to two additional lysines. This second generation molecule has four free amino groups each of which can be covalently linked to an additional lysine to form a third generation molecule with eight free amino groups. A peptide may be attached to each of these free groups to form an octavalent multiple peptide antigen (MAP). The process can be repeated to form fourth or even higher generations of molecules. With each generation, the number of free amino groups increases geometrically and can be represented by 2.sup.n, where n is the number of the generation. Alternatively, the second generation molecule having four free amino groups can be used to form a tetravalent MAP with four peptides covalently linked to the core. Many other molecules, including, e.g., the amino acids Asp and Glu, both of which have two carboxyl groups and one amino group to produce poly Asp or polyGlu with 2n free carboxyl groups, can be used to form the dendritic core of MAPS.

The term “dendritic polymer” is sometimes used herein to define a product of the disclosure. The term includes carrier molecules which are sufficiently large to be regarded as polymers as well as those which may contain as few as three monomers.

The chemistry for synthesizing dendritic polymers is known and available. With amino acids, the chemistry for blocking functional groups which should not react and then removing the blocking groups when it is desired that the functional groups should react has been described in detail in numerous patents and scientific publications. The dendritic polymers and the entire MAP can be produced on a resin and then removed from the polymer. Ammonia or ethylenediamine may be utilized as the core molecule. In this procedure, the core molecule is reacted with an acrylate ester and the ester groups removed by hydrolysis. The resulting first generation molecules contain three free carboxyl groups in the case of ammonia and four free carboxyl groups when ethylenediamine is employed. The dendritic polymer may be further extended with ethylenediamine followed by another acrylic ester monomer, and repeats the sequence until the desired molecular weight was attained. It is readily apparent to one skilled in the art, that each branch of the dendritic polymer can be lengthened by any of a number of selected procedures. For example, each branch can be extended by multiple reactions with Lys molecules.

Some important features of the dendritic polymer as an immunogenic carrier are that the precise structure is known, there are no “antigenic” contaminants or those that irritate tissue or provoke other undesirable reactions. The precise concentration of the reconstituted RBM polypeptide of the disclosure is known; and is symmetrically distributed on the carrier, and the carrier can be utilized as a base for more than one reconstituted RBM polypeptide so that multivalent immunogens or vaccines can be produced.

When the MAPS is to be employed to produce a vaccine or immunogenic composition, it is preferred that the core molecule of the dendrimer be a naturally occurring amino acid such as Lys so that it can be properly metabolized. However, non-natural amino acids residues may be also employed. The amino acids used in building the core molecule can be in either the D or L-form.

In brief, in manufacturing vaccine products it is important to have a good understanding of what factors can impact the safety, efficacy, and stability of the formulation all along the development path. The main phases in this process are: biophysical characterization of the antigen, evaluation of stabilizers, investigation of antigen interactions with adjuvants, evaluation of product contact materials such as sterile filter membranes, and monitoring stability both in real time and under accelerated conditions.

Biophysical Characterization Phase refers to evaluation of the physical characteristics of the antigen. i.e. understanding how parameters such as pH, buffer species, and ionic strength impact the folded state of the antigen as well as the propensity of the antigen to aggregate. Knowing how characteristics of the formulation will impact physical stability of the antigen will aid selection of appropriate excipients during the development process.

Empirical Phase refers to initiation of preformulation studies for the systematic development of a vaccine formulation. A logical place to start, is understanding how the physical stability of the antigen is impacted by changes in pH and temperature. The pH of the formulation can impact both the physical stability of the antigen, such as whether the antigen maintains the appropriate folding and if the antigen will aggregate, as well as the chemical stability of the antigen. The pH can impact the chemical degradation rate of many mechanisms of degradation such as hydrolysis, oxidation, and deamidation. The Empirical Phase Diagram offers a convenient way to display how the physical stability of an antigen is impacted with changes in pH and temperature. Generally in this approach, characterization data are taken from various spectroscopic techniques such as second derivative UV/Vis, intrinsic fluorescence, extrinsic fluorescence, and circular dichroism are combined and transformed into data vectors to construct the empirical phase diagram. In addition to pH evaluation, the Empirical Phase Diagram approach can be utilized to determine the impact of other variables on antigen stability like buffer type and concentration, ionic strength, and impact of product contact material.

Evaluation of Stabilizers refers to optimization of formulation parameters such as pH, ionic strength, and buffer species may not prove to be enough to stabilize an antigen for the typically desired 3-year shelflife of vaccine products. In this case stabilizing excipients need to be investigated for incorporation into the vaccine formulation. Evaluation of antigen stabilizers typically begins with investigation of generally regarded as safe (GRAS) excipients.

By utilizing GRAS excipients, development may proceed more rapidly as regulatory concerns regarding safety of the formulation excipients will be lower. Since at the early stage of development the primary mechanism of antigen degradation may not be known it is important to evaluate excipients from various classes of stabilizers. Excipient screening such as monitoring of optical density or extrinsic fluorescence can be performed in a 96 well format to allow high-throughput screening of many excipients and excipient combinations at one time.

Correlation of Real-time and Accelerated Stability, refer to analysis of the stability of a formulation under extreme environmental conditions such as high temperatures. Correlation of accelerated stability with real-time data is valuable to support activities such as expiration dating and assessment of the impact of temperature excursions during shipment and storage of the vaccine for clinical trials. When initiating stability studies it is important to understand potential mechanisms of antigen can degradation. In general, physical instability is associated with loss of protein structure and aggregation while common forms of chemical degradation are oxidation and deamidation. In addition to high temperature excursions, it is useful to determine the impact of other factors on formulation stability, such as exposure to environmental stresses such as cycles of freezing and thawing, extended exposure to light, and contact with various storage container materials.

Adjuvants or Adjuvantation refers to enhancement of antigen immunogenicity. A side effect of vaccine antigens becoming more pure as purification technology has advanced is a reduction in the immunogenicity of the antigen. To retain antigen immunogenicity with more highly purified antigens, adjuvants can be incorporated into the vaccine formulation. Adjuvants interact with the immune system through various mechanisms thereby enhancing the immune response. Currently, the most utilized adjuvants in licensed products are aluminum salts and squalene-based oil-in-water emulsions. Aluminum-containing adjuvants, including aluminum hydroxide (AlO(OH) and aluminum phosphate (Al(OH)ₓPO₄)_{y} adjuvants, have along history of use and an excellent safety profile.

Sterile Filtration refers to prevention of microbial contamination of vaccines is an important part of producing a safe vaccine formulation. As vaccines are administered to infants, children, and adults who are generally healthy at the time of injection there is a high level of safety that must be ensured when manufacturing the vaccine product. Typically, this can be achieved through aseptic processing and sterile filtration of the vaccine formulation. However, formulations with aluminum-containing adjuvants cannot be sterilized by filtration due to the particle size of the adjuvant being greater than 0.2.mu.m. Materials used to prepare vaccines with aluminum-containing adjuvants must be sterilized prior to formulation and handled aseptically during the formulation and filling process. Sterile filter membranes are produced with various materials, typical membranes used in vaccine production are cellulose acetate, polyethersulfone and polyvinylidene fluoride.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the disclosure are described in detail, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade. and pressure is at or near atmospheric.

The examples are representative of techniques employed by the inventors in carrying out aspects of the present disclosure. It should be appreciated that while these techniques are exemplary of preferred embodiments for the practice of the disclosure, those of skill in the art, in light of the present disclosure, will recognize that numerous modifications can be made without departing from the spirit and intended scope of the disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

Disclosed and described, it is to be understood that this disclosure is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present disclosure will be limited only by the appended claims and equivalents thereof

### Pharmaceutical Compositions

Pharmaceutical compositions disclosed herein may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin may be exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; the MONAQUAT™ series (Mona Industries, Inc., Paterson, N.J.), polyethyl glycol, polypropyl glycol, and copolymers of ethylene and propylene glycol (e.g., Pluronics, PF68 etc).

Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Compositions described herein may be formulated for controlled or sustained delivery in a manner that provides local concentration of the product (e.g., bolus, depot effect) and/or increased stability or half-life in a particular local environment. The compositions may comprise the formulation of polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents. See, for example, the description of controlled release porous polymeric microparticles for the delivery of pharmaceutical compositions in WO 93/15722.

Suitable materials for this purpose may include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals.

Formulations suitable for intramuscular, subcutaneous, peritumoral, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent may be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. The pharmaceutical composition described herein can be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

Alternatively or additionally, the compositions may be administered locally via implantation into the affected area of a membrane, sponge, or other appropriate material on to which a therapeutic agent disclosed herein has been absorbed or encapsulated. Where an implantation device is used, the device may be implanted into any suitable tissue or organ, and delivery of the therapeutic agent, nucleic acid, or vector disclosed herein may be directly through the device via bolus, or via continuous administration, or via catheter using continuous infusion.

Certain formulations comprising a therapeutic agent disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes. vegetable oils, lubricants, suspending agents, tablet disintegrating agents, and binders also may be employed.

Suitable and/or preferred pharmaceutical formulations may be determined in view of the present disclosure and general knowledge of formulation technology, depending upon the intended route of administration, delivery format, and desired dosage. As a non-limiting example, an effective dose may be calculated according to patient body weight, body surface area, or organ size.

In some embodiments, the pharmaceutical composition is formulated for injectable administration. In some embodiments, the methods comprise injecting the pharmaceutical composition. In some embodiments, the methods comprise administering the pharmaceutical composition in a liquid form via intraocular injection. In some embodiments, the methods comprise administering the pharmaceutical composition in a liquid form via periocular injection. In some embodiments, the methods comprise administering the pharmaceutical composition in a liquid form via intravitreal injection. While some of these modes of administration may not be appealing to the subject (e.g. intravitreal injection), they may be most effective at penetrating barriers of the eye, and the therapeutic agent may be least likely to be washed away by tears or blinking as compared to eye drops, which offer convenience and low affordability.

Without further elaboration, it is believed that one skilled in the art, using the preceding description, can utilize the present disclosure to the fullest extent. The following examples are illustrative only, and not limiting of the remainder of the disclosure in any way whatsoever.

### EXAMPLES—MATERIALS AND METHODS

### Bacterial Strains and Cell Lines

All E. coli strains were cultured in Lysogeny broth (LB) medium (1% w/v Tryptone, 0.5% w/v yeast extract and 1% w/v NaCl) using a non-humidified shaker at 37° C. Spodoptera frugiperda (Sf9) cells and Trichoplusia ni (Hi5) cells were individually maintained in the SIM SF medium and the SIM HF medium (Sino Biological, Beijing, China) using anon-humidified shaker at 27° C. HEK293T and Huh-7 cells were maintained in Dulbecco's modified Eagle's medium (DMEM) supplemented with 10% fetal bovine serum (FBS), 100 units of Penicillin and 0.1 mg/ml of Streptomycin with 5% CO₂ at 37° C.

### Gene Cloning, Protein Expression and Purification

The spike S protein RBD of SARS-CoV-2 for antigenicity evaluation was expressed using the Bac-to-Bac baculovirus expression system (Invitrogen). The coding sequence (codon optimized for insect cells) for the RBD region, which spans residues 319-545 of the spike of the SARS-CoV-2 Wuhan-Hu-1 isolate (accession number MN908947), were synthesized by Convenience Biology Corporation (Changzhou, Zhejiang Province, China). For gene cloning, a previously described gp67 signal peptide sequence¹³ was first incorporated into the pFastBac1 vector via the BamH-I and EcoR-I restriction sites. The RBD gene was then sub-cloned into the modified vector via the EcoR-I and Hind-III sites. In addition, an 8×His tag was further added to the protein C terminus to facilitate protein purification. The sequencing-verified plasmid was subsequently transformed into E. coli DH10b cells to generate the recombinant bacmids.

For protein expression, the bacmid was first transfected into Sf9 insect cells using LipoInsect Transfection Regent (Beyotime Biotechnology, Shanghai, China) according to the manufacturer's instructions. The cell culture supernatants, which contain the packaged recombinant baculoviruses, were harvested about 72 hours post transfection. The baculovirus was then passaged in Sf9 cells for 2-3 times before used for protein production in Hi5 cells.

For protein purification, the culture supernatants from the Hi5 cells were collected about 72 hours after infection and passed through a 5-ml HisTrap excel column (GE Healthcare, Shanghai, China) for primary purification. The recovered proteins were further purified on a Superdex 200 Increase 10/300 GL column (GE Healthcare). Finally, the proteins were exchanged into a buffer consisting of 20 mM Tris-HCl (pH 8.0) and 150 mM NaCl for further use. The purity of the protein was determined by SD S-PAGE and visualized by staining with Coomassie blue and by western blotting using the rabbit anti-RBD antibody (Sino Biological).

### LC-MS/MS Analysis to Identify Glycosylation Sites

For MS test, the protein was first trypsin-digested as previously described^{24,25}. In brief, the purified RBD protein was precipitated with 4 volumes of pre-cooled acetone at −20° C. overnight. The protein pellets were collected with centrifugation at 20,000 g for 10 min. After drying on ice, the protein pellets were dissolved in a denaturing buffer (5% 8 M urea in 50 mM NH₄HCO₃, v/v). The proteins were reduced with 20 mM DTT at 55° C. for 60 min, and alkylated with 55 mM iodoacetamide in the dark at room temperature for an additional 30 min. After carbamidomethylation, the proteins were digested with trypsin (1:50 w/w) at 37° C. overnight.

After being desalted with C18 ZipTip (Millipore) according to the manufacturer's instructions, the digested peptides were analyzed by LC-MS/MS using an EASY-nano-LC 1200 coupled to a Q-Exactive HF-X (Thermo Scientific) without the trap column. Specifically, the peptide samples were loaded onto an in-house packed reversed-phase C18 analytical column (30 cm length×360 μm OD×75 μm ID, 3 μm particle, DIKMA) and were separated at a flow rate of 330 nL/min. The column oven temperature was 60° C. Buffer A was 0.2% formic acid in water, and Buffer B was 80% ACN with 0.2% formic acid. A two hours gradient was applied with 3% B for 6 min, 3-48% B for 100 min, 48-100% B for 6 min, 100% B for 6 min, and 2% B for the last 2 min²⁶. MS spectra were acquired with m/z 700-2000 and a resolution of 60,000 at m/z 200. The automatic gain control (AGC) was set at 2e5 with maximum fill time of 50 ms. For MS/MS scans, the Top 20 most intense parent ions were selected with a 2.0 m/z isolation window and fragmented using HCD with normalized collision energies of 20%/30%/30%27. The AGC value for MS/MS was set to a target value of 5e5, with a resolution of 3,000 and a maximum fill time of 250 ms. Parent ions with a charge state of z=1, 8 or unassigned were excluded and the intensity threshold was 3.3e4. The dynamic exclusion period was 20 s. The temperature of the ion transfer capillary was set to 280° C. The spray voltage was set to 2.8 kV.

For the identification of intact N-glycopeptides, all the raw files were searched with GPSeeker, developed by Tian's group¹⁵. In order to search matching precursor and fragment ions, the isotope peak abundance cutoff (IPACO), isotope peak mass-to-charge ratio (m/z) deviation (IPMD), and isotope peak abundance deviation (IPAD) were respectively set to 40%, 20 ppm and 50%. The search of intact N-glycopeptide spectrum matches (GPSMs) included the following parameters: Y1 ion, Top4; the minimum percentage of matched fragment ions of the peptide backbone, ≥10%; the minimum matched fragment ion of the N-glycan moiety, ≥1; TopN hits, N=2 with Top1 hit(s) having the lowest P score; G-bracket, ≥1; and GF Score, ≥1. G-bracket for a given N-glycosite is defined as the number of peptide backbone b/y fragment ion pairs each of which can independently localize the N-glycosite. GF score for a given N-glycan sequence structure is defined as the number of structure-diagnostic fragment ions each of which can independently distinguish the structure from all the other putative structures with the same monosaccharide composition. After DB search of all the raw datasets, GPSMs were combined and intact N-glycopeptides with the lowest P score were chosen as the final IDs.

The MS Raw files were further searched against the RBD sequence with SEQUEST in Proteome Discoverer (version 2.3; Thermo Fisher Scientific). The precursor peptide mass tolerance was 10 ppm and the fragment ion mass tolerance was 0.02 Da. Two missing cleavages were allowed. Cysteine carbamidomethylation was set as a fixed modification. HexNAc (S/T), Hex(1)HexNAc(1)(S/T) and other potential O-glycosylation modifications with conventional oxidation of methionine and protein N-terminal acetylation were set as variable modifications^{15,24}. Percolator was generated with false discovery rate (FDR) of 1%. All potential O-glycosylation sites were further manually confirmed by the b ions and y ions.

### Surface Plasmon Resonance (SPR) Analysis

Surface plasmon resonance (SPR)-based measurements were performed by Biacore 8K (GE Healthcare, Uppsala, Sweden), as described previously². Human ACE2-Fc was captured to ˜100RU on Sensor Chip Protein A. For kinetic analysis, RBD protein was run across the chip in a 2-fold dilution series (1, 2, 4, 6, 8, 16, 32 nM), with another channel set as control. Each sample bound across the antigen surface was dissociated by HBS-EP+ running buffer for 300 s at a flow rate of 30 μL/min. Regeneration of the sensor chips was performed for 60 s using regeneration buffer (Glycine pH 1.5). The association and dissociation rate constants ka and kd were monitored respectively and the affinity value K_{D} was determined.

### Vaccine Formulation and Mice Vaccinations

Alum-precipitated protein (alum protein) vaccines were prepared as described previously²⁸. Briefly, the purified recombinant RBD protein at the different concentrations was added and incubated with mixing with aluminum hydroxide gel for one hour at 5° C. The different formulations were prepared with the concentrations of 1-100 μg/ml for protein and 1.21 mg/ml for aluminum hydroxide gel.

BALB/c and C57BL/6 mice at 6 to 8 weeks of age were injected intramuscularly with different doses (0.1-20 μg per mouse) of recombinant RBD and different intervals. For example, the mice were immunized with a single injection on Day 0 and collected sera on day 7, or with two vaccinations on day 0, day 7 and collected sera on day 21, compared with two doses on day 0, day 14 and collected sera on day 21. Also, we also investigated the third vaccine on day 21 or longer. Additional control animals were injected with aluminum hydroxide adjuvant [Al(OH)3], recombinant RBD or PBS alone.

Pre-immune sera also were collected before starting the immunization and the sera were collected 7 days after each boost. Also, we also immunized the transgenic hACE2 mice with RBD vaccine and found that the mice had similar level of the antibodies against RBD protein, compared with the wild type mice (FIG. 8).

Sera were kept at 4° C. before use. Also, in an attempt to find the pathways through which our recombinant RBD may activate, recombinant RBD vaccine was injected into genetic deficient mice, Cd4^{-/-}, Cd8a^{-/-}, Casp1^{-/-}, Sting1^{-/-}, Tlr2^{-/-}, Tlr4^{-/-} (all from Jackson Laboratory), Nlrp3^{-/-} (from Genentech), and Il-1β^{-/-} (Tokyo University of Science).

### Identification of Serum Antibody Against S Protein RBD in Patients and in Mice Using an ELISA Assay

Blood samples were collected from the retro-orbital plexus of mice after each antigen boost. After coagulation at room temperature for 1-2 h, blood samples were spun in a centrifuge, 3000 rpm/min for 10 min at 4^{a}. The upper serum layer was collected and stored at −20^{a}. Recombinant RBD or S2 protein as a control was used to coat the flat-bottom 96-well plates (Thermo Scientific NUNC-MaxiSorp) at a final concentration of 1 μg/ml in 50 mM carbonate coating buffer (pH 9.6) at 4° C. overnight. The following day, plates were washed 3 times with phosphate-buffered saline (PBS) containing 0.1% Tween 20 (PBST), and blocking solution containing 1% BSA in PBST was added, followed by 1 h incubation at room temperature. Serially diluted mouse sera were added and incubated at 37° C. for 1 h, and then washed the plates 3 times with PBST. Antibodies including goat anti-mouse IgG horseradish peroxidase (HRP)-conjugated antibody, or anti-mouse IgG1/IgM HRP-conjugated antibody were diluted 1/5000 in blocking solution and added to wells (100 μl/well). After incubation for 1 h at room temperature, the plates were washed 5 times with PBST and developed with 3,3′,5,5′-tetramethylbiphenyldiamine (TMB) for 10 min. The reactions were stopped with 50 μl/well of 1.0 M H₂SO₄ stop solution. The absorbance was measured on a microplate reader at 450 nm (A450). To measure the titer of RBD-specific antibodies induced by recombinant proteins, serum samples were serially diluted and measured by titration.

To investigate the potential immunogenicity of S protein RBD as vaccines in human, serum samples were collected from 16 patients infected with SARS-CoV-2 and 20 healthy donors detected with ELISA in similar way mentioned above. Briefly, the recombinant protein was used to coat 96-well microtiter plates, After blocking with 1% BSA, 1:5 diluted sera were added and incubated, followed by four washes Bound Abs were detected with HRP-conjugated antibody (anti-human IgG/IgM antibody) at 1/2000 dilution. For the detection of IgM, serum samples were added to IgG sorbents and collect the supernatant for further detection centrifugation. All patients with COVID-19 were confirmed by RT-PCR using a 2019-nCoV nucleic acid detection kit.

To investigate cell-mediated immune response, mice immunized with S protein RBD or PBS were sacrificed to isolate lymphocytes which were applied for IL-4, and IFNγ ELISA assay. Briefly, the lymphocytes isolated from the spleens of the immunized mice or mice treated by PBS alone were cultured in RPMI medium 1640 supplied with 10% (vol/vol) FBS, 100 U/ml penicillin, 100 μg/ml streptomycin, 1 mM pyruvate (all from Gibco), 50 μM β-mercaptoethanol, and 20 U/ml IL-2 (all from Sigma-Aldrich). Simultaneously, 1 μg/ml RBD protein was added to activate cells. These cells (1×10⁶ per well) were incubated for 72 h at 37^{a}. Cells cultured without RBD protein were used as negative control. The supernatants were collected for ELISA assay for the cytokines. Also, the potential memory lymphocytes against the recombinant RBD protein were identified by analyzing the phenotypes of these cultured lymphocytes by flow cytometry, as mentioned below in flow cytometry.

### Measurement of the Inhibition of the RBD Binding to Cell Surface ACE2

Binding assay of RBD-Fc with ACE2 was performed by flow cytometry as previously described²⁹. Briefly, ACE2-positive Huh7 cells (a human hepatoma cell line) were collected and washed with Hanks' balanced salt solution. Recombinant SARS-CoV-2 RBD-Fc fusion protein was added to the cells to a final concentration of 0.1 μg/ml in the presence or absence of the sera at a different dilution. The cells were incubated further at room temperature for 30 min. Cells were washed three times with HBSS and then stained with anti-human IgG-FITC conjugate (Sigma-Aldrich, St. Louis, Mo., U.S.) at 1:50 dilution for an additional 30 min. After washing, the cells were fixed with 1% formaldehyde in PBS and processed by the NovoCyte Flow Cytometer (ACEA Biosciences, Inc.), and the results were analyzed with FlowJo V10 software.

### Neutralization of Live SARS-CoV-2 Infection and Pseudovirus

To assess the neutralization of SARS-COV-2 infection, Vero E6 cells (5×10⁴) were seeded in 96-well plates and grown overnight. One hundred TCID50 (50% tissue-culture infectious dose) of SARS-CoV-2) was preincubated with an equal volume of diluted sera before addition to cells. After incubation at 37° C. for 1 h, the mixture was added to Vero E6 cells. On day 3 after infection, the cytopathic effect (CPE) was recorded under microscope and the neutralizing titers of the dilutions of sera resulting in complete or EC 50 inhibition were calculated³⁰.

A neutralization assay based on the pseudovirus was performed by measuring infection of ACE2-transfected 293T (293T/ACE2) cells as previously described^{19,23,31}, Briefly, EGFP-expressing pseudotype viruses were produced by co-transfecting with a plasmid encoding codon-optimized SARS-CoV-2 S protein, a pLenti-EGFP vector and a gag/pol expression plasmid. Supernatants containing pseudovirus were harvest 48 h post-transfection and preincubated with the sera from the immunized mouse at various dilutions and control sera. After incubated for 1 hour at 37° C., the mixture was added to ACE2-transfected 293T (293T/ACE2) cells to detect viral infectivity. Media was changed the following day and 48 h after infection, EGFP expression in infected cell was determined by fluorescent microscopy and flow cytometry.

Also, the data on 50% neutralization activities in our FIG. 5b, FIG. 6b and FIG. 7 were performed as described³². Infection of HEK293 cells expressing human ACE2 by SARS-CoV-2 pseudovirus was determined in the presence of mice, rabbit, or monkey sera at a series of 3-fold dilutions. In regard to SARS-CoV-2 pseudovirus that express spike protein, its backbone was provided by VSV G pseudotype virus (G*ΔG-VSV) that packages expression cassettes for firefly luciferase instead of VSV-G in the VSV genome³².

Challenge of the Non-Human Primates (Macaca mulatta) with Live SARS-CoV-2

Twelve adult non-human primates (Macaca mulatta) (5-9 years old) were used for the challenge study with live SARS-CoV-2 and assigned into the following groups: (a) the group immunized with 40 μg RBD protein with Al(OH)₃ adjuvant per dose (n=4), (b) the group immunized with 20 μg with Al(OH)₃ per dose (n=3), (c) with PBS (control treatment, n=3), and (d) with Al(OH)₃ adjuvant alone (n=2). Non-human primates were immunized with two injections on day 0, day 7 via intramuscular route and then challenged with SARS-CoV-2 intranasally (0.5 ml, 10⁶ pfu/ml) on day 28 after the first vaccination. A quantitative real-time reverse transcription-PCR (qRT-PCR) was employed to measure viral genomic RNA (gRNA), and viral subgenomic RNA (sgRNA, indicative of virus replication). Viral load in the lung tissues, throat swabs and anal swabs were measured by qRT-PCR. The primer and probe sequences used were derived from NP gene (Forward: 5′-GGGGAACTTCTCCTGCTAGAAT-3′, Reverse: 5′-CAGACATTTTGCTCTCAAGCTG-3′, Probe: 5′-FAM-TTGCTGCTGCTTGACAGATT-TAMRA-3′), according to the sequences recommended by WHO and China CDC. SARS-CoV-2 E gene subgenomic mRNA (sgmRNA), indicative of virus replication, was assessed by RT-PCR using an approach similar to previously described³³, based on the primer and probe sequences (Forward: 5′-GCTAGAGAACATCTAGACAAGAG-3′, Reverse: 5′-ACACACGCATGACGACGTTATA-3′, Probe: 5′-FAM-TGTGATCGGTAGGAATGACGCGAAGC-Quencher-3′).

For paraffin-embedded sections, tissues were collected and fixed in 10% neutral buffered formalin, embedded in paraffin, and 5 μm sections were prepared for standard hematoxylin and eosin (H&E) staining.

Adoptive Therapy of Splenic T Cells or Immune Sera in the Mice Challenged with Live SARS-CoV-2

All procedures involved in the animal study were reviewed and approved by the Institutional Animal Use and Care Committee of the Institute of Laboratory Animal Science, Peking Union Medical College. Mice studies were performed in an animal bio-safety level 3 (ABSL3) facility using HEPA-filtered isolators. The animal experiments of the infection of SARS-CoV-2 were performed by specific pathogen-free transgenic hACE2 mice established by the Institute of Laboratory Animal Science, Peking Union Medical College, China. Transgenic mice were generated by microinjecting a transgene carrying a mouse ACE2 promoter driving the human ACE2 coding sequence into the pronuclei of fertilized ova from ICR mice, as described in detail³⁴. The transgenic hACE2 mice with C57BL/6 background³⁵ were provided by the National Institutes for Food and Drug Control (NIFDC, Beijing, China)

An adoptive therapy of splenic T cells was conducted previously^{24,36}. hACE2 mice with C57BL/6 background received 5×10⁷ splenic T cells isolated from either mouse with same C57BL/6 background 9 days after the third dose of the candidate vaccine or from the mice treated with PBS as a control. The adoptive therapy of the sera was described previously²⁷. The adoptive therapy based on immune sera was performed using 0.1 ml of the pooled sera from the immunized mice at the same time. In addition, hACE2 mice with ICR background received 0.8 ml sera from the mice 7 days after a single dose of the vaccine and challenged with live SARS-CoV-2. The mice were sacrificed 5 days after the challenge with live virus, and viral loads in lung tissues, lung histopathological changes, and body weight change were evaluated. Viral load in the lung tissues were measured by qRT-PCR. Sections were stained by Hematoxylin and Eosin (H&E) and evaluated under light microscopy.

### Flow Cytometry

T cells was evaluated with flow cytometry as previously described³⁷. Mice immunized with S protein RBD or PBS were sacrificed to collect lymphocytes. The lymphocytes were cultured in RPMI medium 1640 supplied with 10% (vol/vol) FBS, 100 U/ml penicillin, 100 μg/ml streptomycin, 1 mM pyruvate (all from Gibco), 50 μM β-mercaptoethanol, 20 U/ml IL-2 (all from Sigma-Aldrich) for 72 h. At the same time, 1 μg/ml S protein RBD was added to activate cells. Brefeldin A (BD Biosciences) was administrated 4-6 h before staining to block intracellular cytokine secretion. Cells were then washed in PBS (Gibco) and stained for 30 min at 4° C. with anti-CD8, anti-CD4, anti-CD44, anti-B220, anti-MHCII (all from BioLegend). Afterwards, cells were fixed and permeabilized to facilitate intracellular staining with anti-IFNγ, and anti-IL-4 (all from BioLegend). Flow cytometry data were acquired on a NovoCyte Flow Cytometer (ACEA Biosciences, Inc.) and analyzed using FlowJo V10 software.

### Statistical Analysis

Statistical analyses were performed using the Prism 8.0 (GraphPad Software). Comparisons among multiple groups across multiple time points were performed using a two-way ANOVA test with Tukey's multiple comparison post test. Comparisons among multiple groups were performed using a one-way ANOVA test followed by Tukey's multiple comparison post test. Comparisons between two groups were performed using an unpaired Student's t tests. P-values of <0.05 were considered significant. *P<0.05, **P<0.01, ***P<0.001, ****P<0.0001. NS, no significance.

### EXAMPLES—RESULTS

The examples and embodiments described herein are for illustrative purposes only and are not intended to limit the scope of the claims provided herein. Various modifications or changes suggested to persons skilled in the art are to be included within the spirit and purview of this application and scope of the appended claims.

### Example 1—Characterization of the Recombinant SARS-CoV-2 S RBD Protein

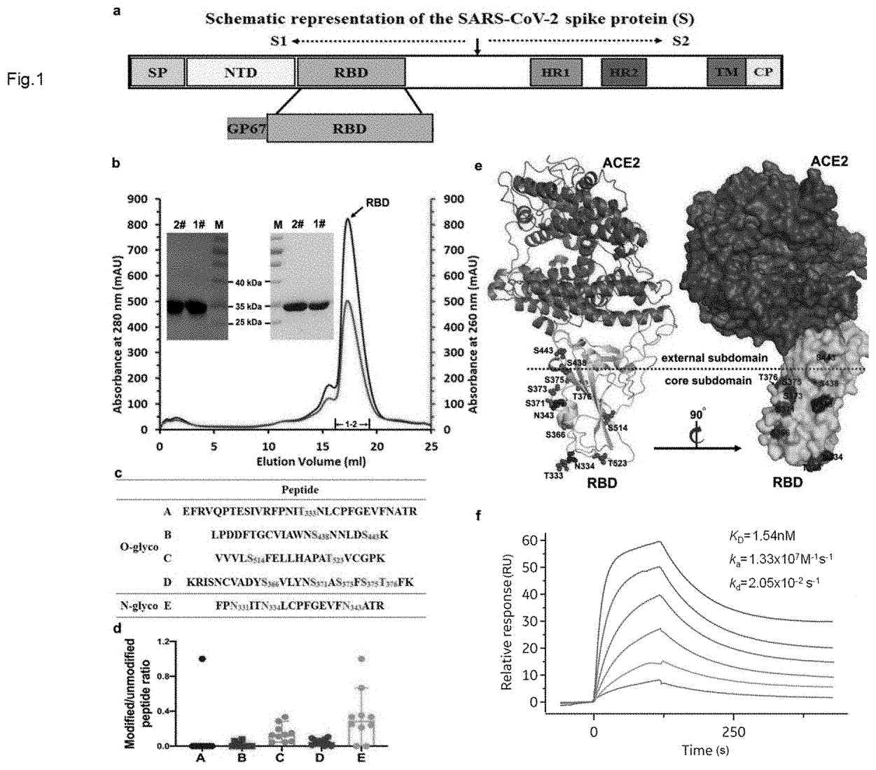

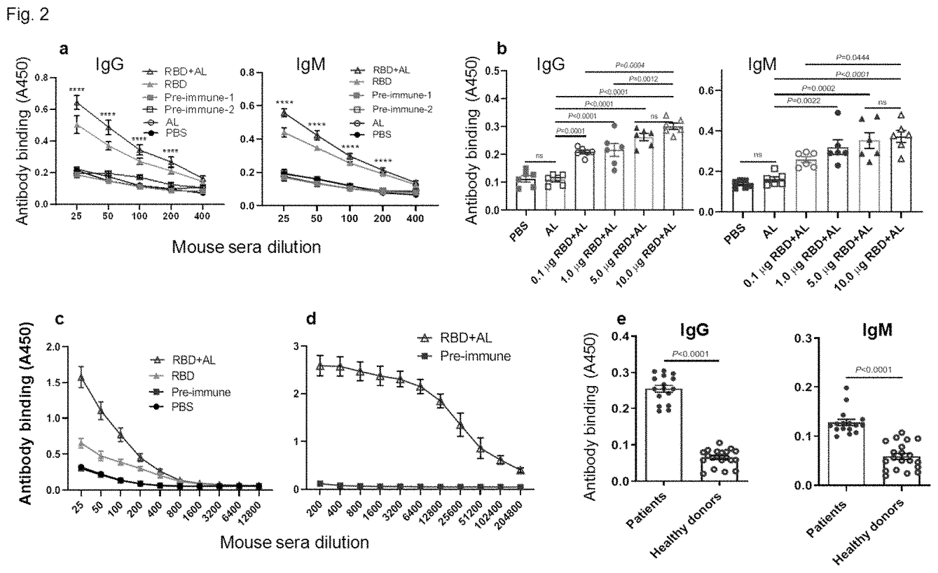

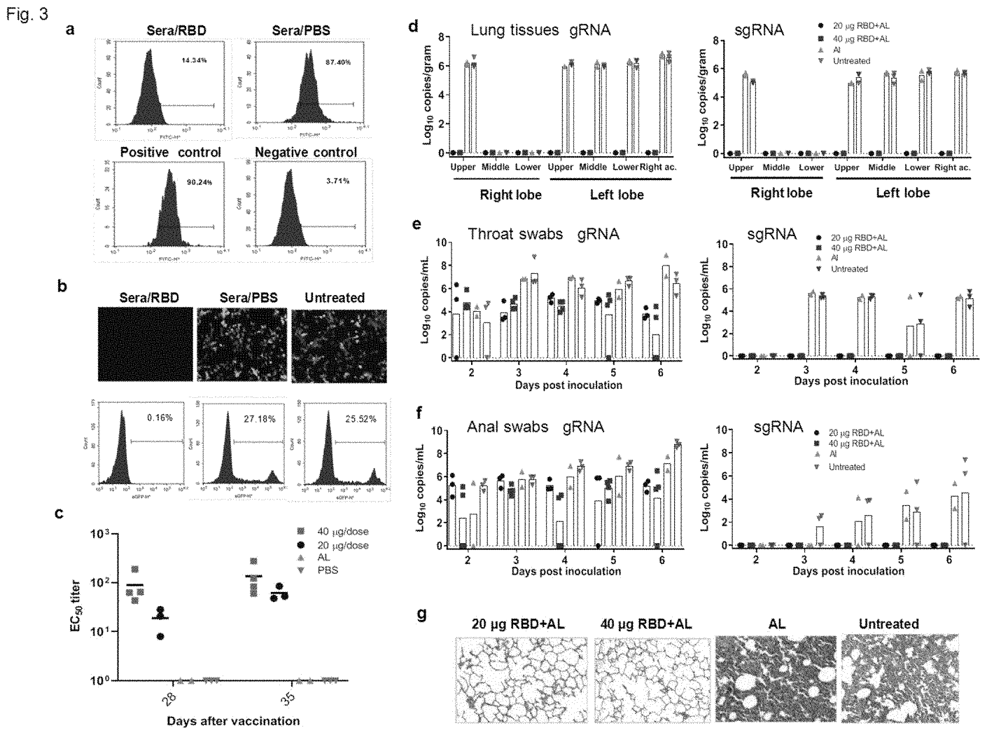

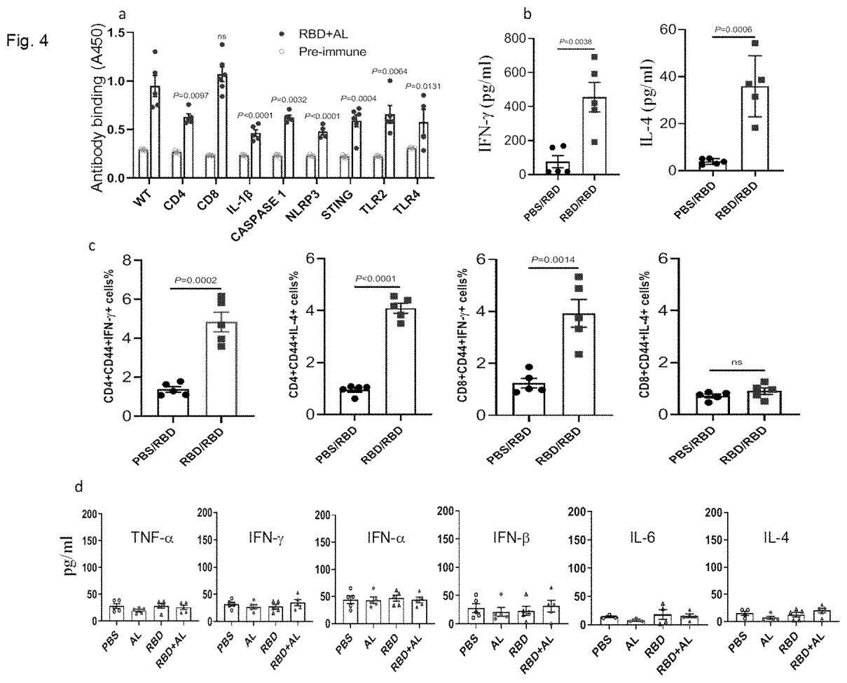

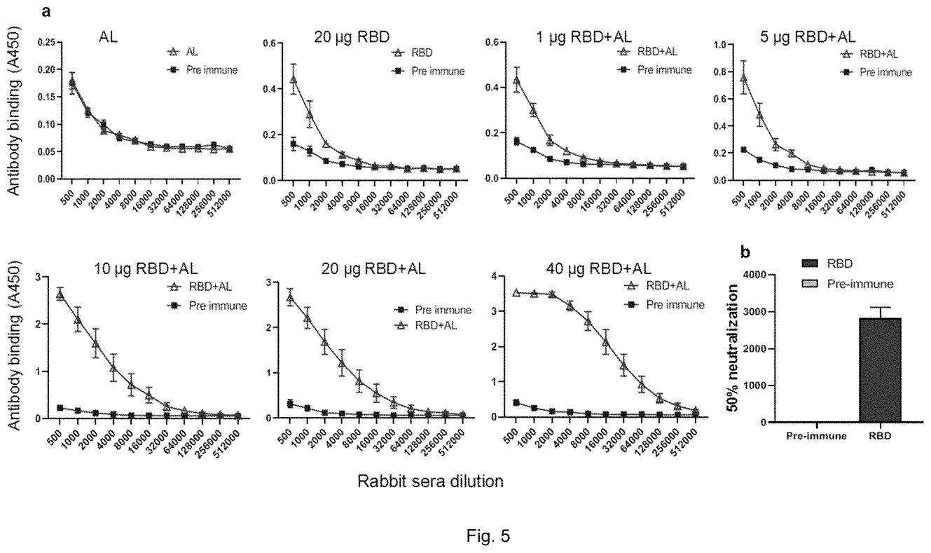

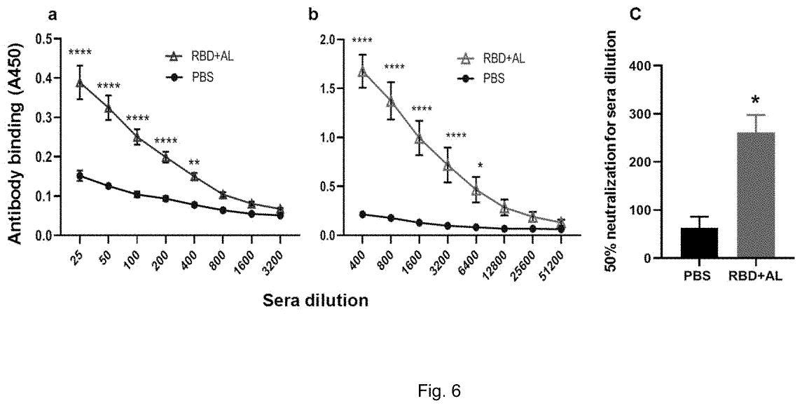

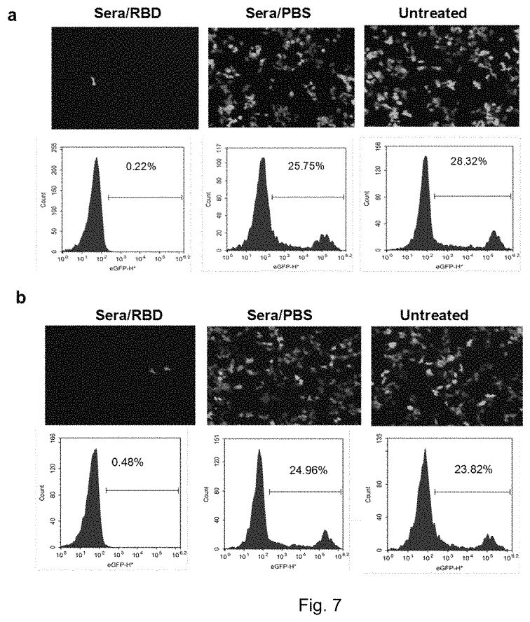

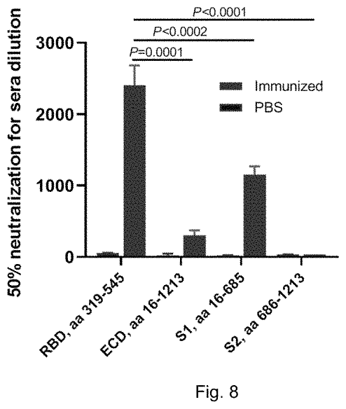

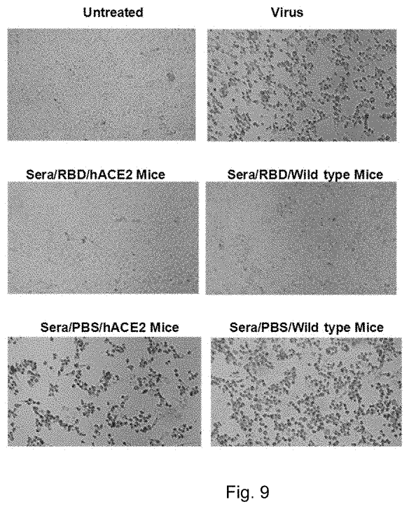

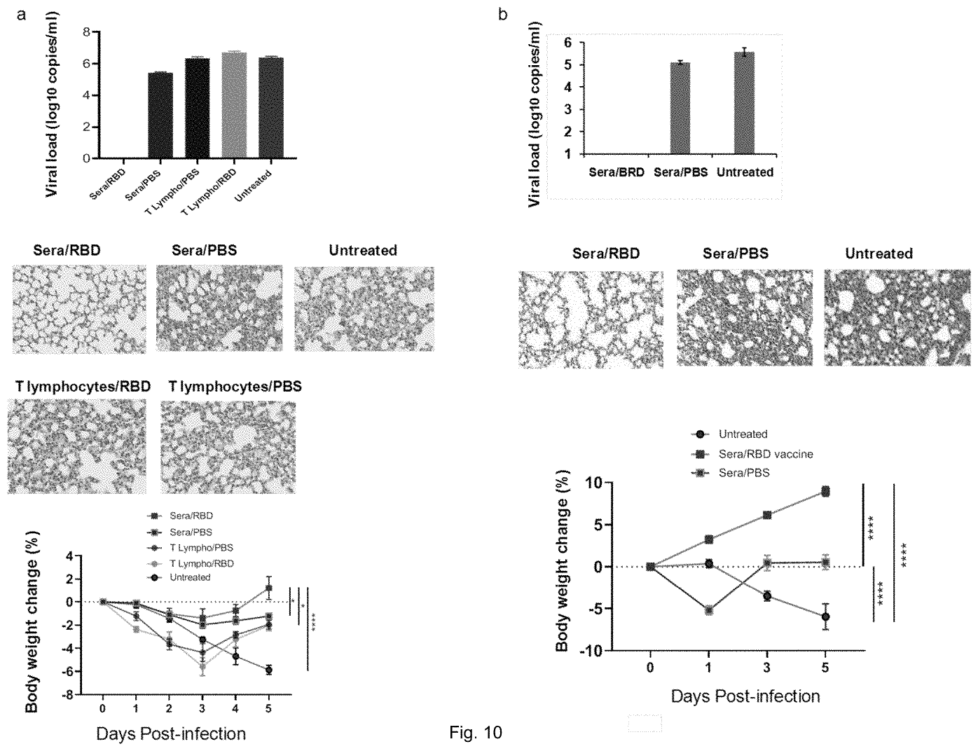

The recombinant receptor-binding domain (RBD) protein of SARS-CoV-2 spike protein was prepared using insect cells and the Bac-to-Bac Baculovirus Expression System as described previously^{13,14}. To ensure efficient protein secretion, a GP67 signal peptide sequence was added to the N-terminus (FIG. 1a). Following protein expression, recombinant RBD was harvested from the culture supernatant via Ni-NTA affinity chromatography. Further purification of the protein using gel filtration revealed a symmetric elution peak (FIG. 1b), indicating a high level of homogeneity of the protein in solution. The pooled samples were further analyzed by SDS-PAGE and western blotting, which revealed a purity of over 98% (FIG. 1b).

The apparent molecular weight of the purified RBD protein was determined to be 34 kDa, which was approximately ¼ larger than that calculated from the molecular weight of the RBD amino acid sequence alone (about 27 kDa). This result suggested that there was post-translational glycosylation of the protein. Experiments were then performed to identify the glycosylated sites in the expressed SARS-CoV-2 RBD. The purified protein was digested with trypsin and analyzed by mass spectrometry (MS). The intact N-glycopeptides and glycans were analyzed using the GPSeeker software^{14,15} Overall, three N-glycosylation sites on asparagine were identified (FIG. 1c). Among them, there were 17 glycan moieties on N331, 12 glycans on N334 and 19 glycans on N343. The O-glycosylation sites was also evaluated by analyzing the MS results using SEQUEST in Proteome Discoverer (version 2.3). Some well-known O-linked glycans such as HexNAc and Hex(1)HexNAc(1) were searched as potential variable modifications^{15,16}. In total, ten O-glycosylation sites were identified, including seven serine (S366, S371, S373, S375, S438, S443 and S514) and three threonine residues (T333, T376 and T523; FIG. 1c). To determine the abundance of glycosylation, the number of MS/MS spectra of glycosylated peptides and their corresponding unmodified peptides were both determined. A much higher degree of N-glycosylation than O-glycosylation was observed (FIG. 1d).

These identified glycosylation sites were further mapped on the recently solved complex structure of the SARS-CoV-2 RBD as bound to its receptor ACE2. Overall, majority of the sites were located on the core subdomain of RBD (FIG. 1e). In addition, all the sites were found to be distant from the bound ACE2 molecule (FIG. 1e), indicating that the decorated glycans on these sites may not interfere receptor recognition/binding.

The functional binding of our recombinant RBD protein with the ACE2 receptor was confirmed via surface plasmon resonance Biacore. In concordance with a recent study², potent interactions with a typical slow-on/slow-off binding kinetics between our protein and ACE2 were observed. The binding affinity was calculated to be about 1.54 nM in dissociation constant (K_{D}), with kₐ of 1.33×10⁷ M⁻¹ S⁻¹ and k_{d} of 2.05×10⁻² S⁻¹ (FIG. 1f). This finding showed that our recombinant RBD is binding to ACE2 with a high affinity, suggesting that our RBD domain protein is likely a good reflection of the native conformation.

### Example 2—Identification of Serum Antibody Against S Protein RBD in Patients and Animal Models

First, alum-precipitation was selected to serve as a vaccine adjuvant based on its long record of effectiveness and safety^{17,18}. In this study, all vaccine preparations were prepared by the addition of aluminum hydroxide gel to various proteins, resulting in alum-precipitated protein vaccine candidates.

Mice were immunized with different dose range (0.1 to 20 μg) and regimen. For example, mice were immunized with a single injection on Day 0 and collected sera on day 7; or with two vaccinations on day 0, day 7 and collected sera on day 21, with two doses on day 0, day 14 and sera collected on day 21. In some experiments, a third dose on Day 21 was also given. To assess the humoral immune responses induced by the recombinant RBD, enzyme-linked immunosorbent assay (ELISA) for RBD-specific antibodies was employed. Given the urgent need of an effective vaccine globally, special attention was provided to the early antibody response and its ability to neutralize SARS-CoV-2.

Sera obtained on day 7 after the first does of the candidate vaccine already showed elevated IgG and IgM responses to the recombinant RBD (FIG. 2a). In contrast, the sera from pre-immunization and those from PBS and Alum gel controls had only background-level antibody responses. Furthermore, the antibody reaction was dose-dependent, and could be induced with a very low dose of the vaccinated protein (0.1 μg/mice) 7 days after the administration (FIG. 2b). The recombinant RBD protein alone was already effective in inducing specific antibodies production, but the addition of the Alum adjuvant could significantly enhance the induction with a higher level of specific antibodies by day 7 (FIG. 2a) and even more by day 21 (FIG. 2c). The sera obtained 9 days after the third vaccination (booster dose given on day 21) showed a very strong specific antibody response in the mice (FIG. 2d), with a positive reaction at a dilution of 1:204,800.

The antibody reaction was also dose-dependent (FIG. 2b). The dose-dependent response was also observed in rabbits, and a good level of specific antibody could be induced with a low dose at 1 μg/rabbit per injection and with three doses of the candidate vaccines given (n=42 rabbits, FIG. 5a). Importantly, we also tested the viral neutralization activity of the sera as challenged in vitro with a pseudovirus and a high level of activity was observed (see below, and Extended FIG. 1b).

We also tested our candidate vaccine in non-human primates (Macaca mulatta). Ten monkeys were vaccinated at day 0 and Day 7 and immune sera were obtained at day 7 after each vaccination with our candidate vaccine or pre-immunization was used as a reference. Sera at day 7 and 14 post-immunization showed a significantly elevated IgG responses to the recombinant RBD and had an increased level of neutralizing antibodies against pseudovirus (FIG. 6). Also, the neutralizing antibodies against live SARS-CoV-2 was demonstrated in the vaccination of non-human primates and challenge with live SARS-CoV-2 (see below, FIG. 3 c).

The antigenicity of the recombinant RBD was also tested for seroreactivity in sera obtained from healthy human subjects and patients with COVID-19. Sera from 20 healthy donors and 16 patients with COVID-19 (which were confirmed by reverse transcription polymerase chain reaction RT-PCR nucleic acid detection kit for the detection of SARS-COV-2 sequence) were tested for their IgG and IgM responses to the recombinant RBD. All 16 COVID19 patients showed significant elevated levels of IgG and IgM against the recombinant RBD, compared with the background signal recorded in the healthy donors (FIG. 2e). These 16 patients subsequently recovered from the infection.

These findings indicated that the S-protein RBD domain contained in the candidate vaccine (a) could trigger a good antibody response in rodents, rabbits and non-human primates, (b) in a dose dependent manner in rodents and rabbits, (c) and can trigger an early antibody response with viral neutralizing activity even with one dose of the candidate vaccine, and (d) and similar antibody response was also seen in patients infected with the COVID-19. The next question will then be, is the observed antibody response a good indicator of the viral neutralizing activity, the primary objective of the preventive vaccine.

### Example 3—Functional Characterization of the Sera from the Immunized Animals and the Prevention of the Mice from SARS-CoV-2 Infection

In the next sets of experiments, sera from the immunized animals were tested for its blocking activity of RBD to ACE2 receptor. ACE2-positive Huh7 cells (a human hepatoma cell line) were selected for detecting the RBD-binding activity in a flow-cytometric assay. Fc-conjugated RBD protein was added to the ACE2-positive Huh7 cells in the absence of any sera, followed by incubation with a secondary anti-Human IgG-FITC conjugate. Negative control Huh7 cells in the absence of immune sera had RBD-ACE2-positivity detected in 90.2% of cells (FIG. 3a). With the immunized sera obtained in mice 7 days after one dose (5 μg) of the candidate vaccine, only 14.3-% Huh7 cells were RBD-ACE2 positive. Sera from the mice treated with phosphate buffered saline (PBS) at the same dilution had nearly no inhibitory activity with 87.4-% ACE2 positive cells (FIG. 3a). These findings indicated that the sera from the early vaccination with a single dose (5 μg) in mice could effectively block RBD binding to AEC2 receptor on the cells.

A neutralization assay using pseudovirus is regarded as a sensitive and quantitative method for SARS-CoV and MERS-CoV¹⁹. We constructed a SARS-CoV-2 pseudovirus expressing EGFP, generated by co-transfecting with a plasmid encoding codon-optimized SARS-CoV-2 S protein, a pLenti-EGFP vector and a gag/pol expression plasmid. Immune sera from the non-human primates (Macaca mulatta), mice and rabbits were tested for the neutralizing activity against SARS-CoV-2 pseudovirus in 293T cells expressing ACE2. Immune sera from the non-human primates (Macaca mulatta) 7 days after the first vaccination can block nearly completely the infection by SARS-CoV-2 pseudovirus (FIG. 3b). The pre-immune sera or those from the non-human primates treated with PBS at the same dilution had no inhibitory activity on SARS-CoV-2 pseudovirus infection. Similarly, nearly complete neutralization of SARS-CoV-2 pseudovirus was observed using the immune sera from mice and rabbits 14 days after the first vaccination (FIGS. 7a and b).

The mice were immunized with the recombinant RBD, the extracellular domain protein (ECD), S1-subunit protein (S1) or S2-subunit protein (S2) in the presence of aluminum hydroxide gel on day 0, day 14 and day 21. Sera were collected from the mice after the third vaccination. Recombinant RBD Protein vaccine had a much higher viral neutralization activity with an EC50 at a calculated dilution of 1:2405, compared with the extracellular domain protein (ECD) at a calculated dilution of 1:300, 51-subunit protein (S1) at a calculated dilution of 1:1155 (FIG. 8). No viral neutralization activity was found with S2-subunit protein (S2) vaccine. In addition, the immune sera from both human ACE2 transgenic mice and wild type mice 14 days after the second vaccination showed a similar level of the neutralizing antibodies with a calculated dilution of 1:32 or lower that can completely protect Vero E6 cells from live SARS-CoV-2 infection (FIG. 9). When the immune sera from rabbits immunized with three doses of the vaccine candidate were tested, a viral neutralization activity with an EC50 at a calculated dilution of 1:2826 was observed in rabbits (FIG. 5b).

In the next experiments, we also tested whether the RBD vaccine could block the infection in non-human primates inoculated with live SARS-CoV-2. We immunized non-human primates (Macaca mulatta) with two injections on day 0, day 7 via the intramuscular route with 20 μg or 40 μg per dose and then challenged with live SARS-CoV-2 viruses 28 days after the first vaccination. The control groups included the treatment with PBS or aluminum hydroxide gel alone. Neutralizing antibodies against live SARS-CoV-2 were detected in all vaccinated non-human primates, whereas no neutralizing antibodies were detected in the two control groups (FIG. 3c).

A quantitative real-time reverse transcription-PCR (qRT-PCR) was employed to measure viral genomic RNA (gRNA) and viral subgenomic RNA (sgRNA, indicative of viral replication). Lung tissues of non-human primates were collected on day 7 following challenge to determine the viral replication status. Lung tissues from the control groups showed excessive copies of the viral gRNA and sgRNA in lung tissues. In contrast, no detectable viral gRNA or viral sgRNA was detected in the vaccinated groups with 20 μg and μg of the vaccine with adjuvant given (FIG. 3d). In addition, peak loads of viral gRNA in the throat swabs were observed in the controls (FIG. 3e) 3 days post inoculation (DPI) and these were blocked by the vaccination, with only ˜1.6 and 3.8 parts per million of the viral loads in the vaccinated groups with 20 and 40 μg of vaccines given, respectively, compared with those of the control group. Importantly, no detectable sgRNA in the throat swabs was observed in the vaccinated groups with both 20 μg and 40 μg doses after the challenge (FIG. 3d), whereas high levels of sgRNA, indicative of viral replication, was observed in the control groups. Peak levels of viral gRNA and sgRNA in the anal swabs were observed in the control groups at 5- and 6-days post inoculation, but only a very low level detection in the vaccinated groups and again no detectable sgRNA was detected in the anal swabs in the vaccinated non-human primates in both 20 and 40 μg dose groups (FIG. 3f). These data were consistent that the low gRNA detected was from the high inoculation dose which might be neutralized already and there was no evidence of viral replication as reflected by the absence of detectable sgRNA.

The lung tissues from the two control groups (aluminum hydroxide or PBS alone) revealed typical histopathological changes of viral interstitial pneumonia, a key feature of COVID-19. The microscopic findings included apparent thickened alveolar walls, heavy interstitial infiltrates by mononuclear cells and lymphocytes, congestion, as well as serosanguineous exudates in the alveolar spaces, or diffuse hemorrhage. Type II pneumocyte hyperplasia was also observed. In contrast, non-human primates vaccinated with the RBD vaccine (20 μg or 40 μg) exhibited no significant histopathological changes nor any evidence of focal infiltrate (FIG. 3g).

### Example 4—Assessment of the Immune Cellular Pathways Involved with the RBD Vaccination

Next, the potential pathways through which our recombinant RBD protein vaccine were involved in mounting this immune humoral response were evaluated. Recombinant RBD was injected into Cd4^{-/-}, Cd8a^{-/-}, Sting1^{-/-}, Tlr2^{-/-}, Tlr4^{-/-}, Casp1^{-/-}, Nlrp3^{-/-}, and Il-1β^{-/-} mice. As shown in FIG. 4a, the mice deficient in Cd4^{-/-}, Sting1^{-/-}, Casp1^{-/-}, Nlrp3^{-/-}, and Il-1β^{-/-}, Tlr2^{-/-}, Tlr4^{-/-} showed reduction in the level of IgG induced against the RBD protein, as compared to wild-type mice, while others (Cd8a^{-/-} mice) showed no effect on the level of IgG induction. These findings implied that the NLRP3 inflammasome, Sting, TLR-4, and TLR-2 pathways, and CD4 T lymphocytes are involved in the induction of IgG against RBD as triggered by the candidate RBD vaccine.

Cellular immune responses may be also involved in the clearance of SARS-CoV infection in which both CD4 and CD8 T cells are involved²⁰⁻²³. To address this question, lymphocytes from mice were collected 7 days after the first vaccination and cytokines including IFN-γ and IL-4 produced by the lymphocytes were assayed by ELISA. The lymphocytes isolated from the candidate vaccine-immunized mice induced elevated levels of IFN-γ and IL-4 when stimulated with recombinant RBD (FIG. 4b), while only a background level was detected in the RBD-stimulated lymphocytes derived from the mice treated by a PBS control. The potential memory lymphocytes against the recombinant RBD were also characterized by analyzing the phenotypes of these cultured lymphocytes. Using flow cytometry, the number of the memory lymphocytes, CD4+CD44^{high}+IL-4+, CD4+CD44^{high}+IFN-γ, CD8+CD44^{high}+IFN-γ, were found to be increased in the candidate RBD vaccinated-mice (FIG. 4c).

In order to investigate whether immune sera versus splenic T cells triggered by our vaccine play a role in the protection from live SARS-CoV-2, we performed adoptive therapy of immune sera versus splenic T cells from the vaccinated mice. hACE2 mice with C57BL/6 background received 5×10⁷ splenic T cells isolated from either the mice with same C57BL/6 background after the third dose of the candidate vaccine or from the mice treated with PBS as a control. Adoptive therapy of splenic T cells (CD4+ and CD8+ cells) did not provide the protection from the infection of SARS-CoV-2. By contrast, at the same time, hACE2 mice with C57BL/6 background received 0.1 ml of the pooled sera from the immunized mice after the third dose of the candidate vaccine or from the mice treated with PBS as a control. Adoptive therapy showed no detectable viral replication, no significant histopathological changes as well as no weight loss, compared with those from the mice treated with PBS as a control (FIG. 10a). Even the sera from mice 7 days after a single dose of the vaccine can completely prevent mice from the infection with live SARS-CoV-2 (FIG. 10b). In addition, no evidence of antibody-dependent enhancement or acceleration of pneumonia was observed as no mice that received the RBD-vaccinated immune sera developed any evidence of pneumonia. Our data based on the adoptive therapy of splenic T cells (CD4+ and CD8+ cells) and the level of the antibody in the mice deficient in CD4 or CD8 lymphocytes, indicated that critical role of CD4 lymphocyte response in orchestrating the antibody response against the RBD candidate vaccine in mice.

Although elevated levels of INF-y and IL-4 was observed to be produced by the lymphocytes stimulated by the RBD in vitro, there were no significant increase in any of these two and other inflammatory cytokines in the plasma in these mice (FIG. 4d), indicating that the RBD did not induced a systemic inflammatory reaction, but rather an immune response that is local to the area with exposure to RBD or SARS-CoV-2.

To further address the safety of the candidate vaccine, the potential toxicity of the vaccine was evaluated in the non-human primates (n=50) in compliance with Good Laboratory Practices (GLP). No adverse events were observed including body weight, ruffling of fur, behavioral changes, appetite, etc. No pathologic changes were observed in liver, lung, kidney, spleen, brain, heart, or other tissues on microscopic examination. No changes in serum biochemistry, peripheral blood counts and differentials were noted (data not shown).

### CERTAIN NON-LIMITING FEATURE OF PRESENT DISCLOSURE

The present disclosure showed a few important non-limiting technical features. First, SARS-CoV-2 RBD is the important antigen to serve as a potential vaccine candidate and importantly, the induction of neutralization activity is better than the full-length extracellular protein, and the S1 and S2 domains. Second, the candidate RBD vaccine can induce sufficient level of anti-RBD and neutralizing antibody with even one dose of the candidate vaccine, and three doses of the candidate vaccine can generate a very high level of humoral response. Third, the high level antibodies induced by the candidate RBD vaccine is associated with a significant level of viral neutralization activity and can protect mice from SARS-CoV-2 challenge and demonstrable viral neutralizing activities in rodents, rabbits and monkeys against a SARS-CoV-2 pseudovirus infection in vitro. Fourth, passive transfer of the sera with viral neutralizing activity can also neutralize the viral challenge. Finally, both B-cells and T-cells are involved in orchestrating the good humoral response against the candidate RBD antigen.

SARS-CoV-2 is causing a major pandemic and is an urgent medical problem. The observation that the SARS-CoV-2 protein containing the RBD domain can provoke a higher humoral antiviral neutralization response than the full extra-cellular protein domain and the S1 and S2 domain is important for the scientific community. It highlighted that efforts should be focused on this domain and should form the basis for future immune augmentation efforts.

Certainly, it will be important to compare the humoral response generated by this approach versus the inactivated whole viruses. The fact that commercial scale culture of SARS-CoV-2 is a massive undertaking not without risk may suggest that this recombinant RBD approach may be more technically and commercially feasible to advance for a massive global supply scale.

The demonstration that this candidate RBD vaccine can induce viral neutralizing activity in three different animal species, rodents, rabbits, and non-human primates are encouraging. It is also encouraging to observe (a) that the antibodies shared common binding epitopes from infected patients, (b) that there is a strong viral neutralizing activity associated with this good humoral response, (c) that a simple vaccine adjuvant like Alum can further enhance the immune response, (d) that even one dose of the vaccine can already generated a good level of viral neutralizing activity, and (e) that preliminary toxicology studies showed that this vaccine candidate is safe. All these features are encouraging in supporting the further development of this vaccine candidate. What is intriguing is the early protective viral neutralizing activity observed. Given the current epidemic, whatever is the final vaccination regime, if healthy subject can already develop some type of viral neutralizing activity around 7 days after the first dose (while still following the final vaccination regime as defined clinically), this may have tremendous impact to partially solving the current impact of SARS-COV-2 to the world.

The protection of the mice against SARS-COV-2 challenge was also very important. It provided direct confirmation of the in vivo activity. One would like to ask why the experiments were not being performed as direct challenge, rather than as passive transfer of sera in the current experiments. The reason was that transgenic hACE2 animals are in very short supply globally. In the design of our experiments, we were trying to address two questions at the same time, i.e. whether the candidate vaccine could generate protective viral neutralizing activity in vivo and also whether passive transfer of the immune sera (as passive immunotherapy) would also offer protection against direct SARS-CoV-2 challenge. Our results were clear. The candidate vaccine did generate viral neutralizing activity and importantly, this activity could be transferred passively. This observation can serve as the scientific basis that supports some of the recent suggestions that passive immunotherapy with convalescent serum from COVID-19 patients can prevent the infection and may also help infected patients from reducing the viral load. However, this is an early study and the clinical utility of passive immunotherapy with convalescent sera from COVID-19 patients remained to be addressed in proper controlled clinical studies. The observation that passive immunotherapy can prevent SARS-CoV-2 challenge may lead one to consider that even in infected patients, this approach may reduce the viral load and limited the spread of the virus from cells to cells and therefore, may support the patient's immune system to clear the infection. However, in sick patients, there are many confounding clinical/pathologic variables and only proper controlled clinical studies can address the clinical utility of this approach.

The involvement of both B-cells and T-cells in the coordinated immune response and the cellular pathways involved in the humoral response identified in our study are not surprising. However, such data will provide the scientific community the critical information when they can plan on other experiments that can augment this immune response further. Obviously, when investigators are trying to determine why some patients have more progressive COVID-19 disease than others, these immune factors should form a roadmap for their study involving both host factors on top of viral and other factors involved.

While the principles of this invention have been described in connection with specific embodiments, it can be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

SEQUENCE LISTING
Amino Acid Sequence of S protein of SARS-COV-2:
(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHLDKYFKNHTSPDVDLGD
ISGINASVVNIQKEIDRLNEVAKNLNESLIDLQELGKYEQYIKWPWYIWL
GFIAGLIAIVMVTIMLCCMTSCCSCLKGCCSCGSCCKFDEDDSEPVLKGV
KLHYT
Amino Acid Sequence of MERS-COV Spike Protein:
(SEQ ID NO: 2)
MIHSVFLLMFLLTPTESYVDVGPDSVKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSATIRKIYPAFMLG
SSVGNFSDGKMGRFFNHTLVLLPDGCGTLLRAFYCILEPRSGNHCPAGNS
YTSFATYHTPATDCSDGNYNRNASLNSFKEYFNLRNCTFMYTYNITEDEI
LEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCS
YESFDVESGVYSVSSFEAKPSGSVVEQAEGVECDFSPLLSGTPPQVYNFK
RLVFTNCNYNLTKLLSLFSVNDFTCSQISPAAIASNCYSSLILDYFSYPL
SMKSDLGVSSAGPISQFNYKQSFSNPTCLILATVPHNLTTITKPLKYSYI
NKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQL
GNCVEYSLYGVSGRGVFQNCTAVGVRQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQFNSSYFKLSIPTNFSFGVTQEYIQTTIQ
KVTVDCKQYICNGFQKCEQLLREYGQFCSKINQALHGANLRQDDSVRNLF
ASVKSSQSSPIIPGFGGDFNLTLLEPVSISTGSRSARSAIEDLLFDKVTI
ADPGYMQGYDDCMQQGPASARDLICAQYVAGYKVLPPLMDVNMEAAYTSS
LLGSIAGVGWTAGLSSFAAIPFAQSIFYRLNGVGITQQVLSENQKLIANK
FNQALGAMQTGFTTTNEAFRKVQDAVNNNAQALSKLASELSNTFGAISAS
IGDIIQRLDVLEQDAQIDRLINGRLTTLNAFVAQQLVRSESAALSAQLAK
DKVNECVKAQSKRSGFCGQGTHIVSFVVNAPNGLYFMHVGYYPSNHIEVV
SAYGLCDAANPTNCIAPVNGYFIKTNNTRIVDEWSYTGSSFYSPEPITSL
NTKYVAPQVTYQNISTNLPPPLLGNSTGIDFQDELDEFFKNVSTSIPNFG
SLTQINTTLLDLTYEMLSLQQVVKALNESYIDLKELGNYTYYNKWPWYIW
LGFIAGLVALALCVFFILCCTGCGTNCMGKLKCNRCCDRYEEYDLEPHKV
HVH
Amino Acid Sequence:
(SEQ ID NO: 3)
STGNYNYKYRL
Amino Acid Sequence:
(SEQ ID NO: 4)
FSPDGKPCTPCTPPALNCYWPLNDYGFYTT
Amino Acid Sequence:
(SEQ ID NO: 5)
FSPDGIPCTPCTPPALNCYWPLNDYGFYTT
Amino Acid Sequence of part of S protein of
SARS-COV-2 (amino acid residues 319-545):
(SEQ ID NO: 6)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNFNFNG

1. A method of for inducing an immune response against SARS-CoV-2 in a primate in need thereof, the method comprising the step of administrating to said primate an immunogenetically effective amount of a recombinant polypeptide, wherein the at least a portion of the recombinant polypeptide corresponds to an amino acid residue sequence within the Receptor Binding Domain (RBD) of SARS-CoV-2 spike protein capable of forming a binding interface that interacts with a viral receptor of the primate, wherein the effective amount of the recombinant polypeptide binds to the viral receptor of the primate to induce an immune response against SARS-CoV-2 in the primate.

2. A method for preventing, inhibiting, reducing, eliminating, protecting or delaying the onset of an infection or an infectious clinical condition caused by SARS-CoV-2 in a primate in need thereof, the method comprising the step of administrating to said primate a therapeutically effective amount of a recombinant polypeptide, wherein the at least a portion of the recombinant polypeptide corresponds to an amino acid residue sequence within the Receptor Binding Domain (RBD) of SARS-CoV-2 spike protein capable of forming a binding interface that interacts with a viral receptor of the primate, wherein the effective amount of the recombinant polypeptide binds to the viral receptor of the primate to induce an immune response against SARS-CoV-2 in the primate.

3. The method of claim 2, wherein the infectious clinical condition is COVID-19.

4. The method of claim 1, wherein the amino acid residue sequence within RBD of SARS-CoV-2 spike protein is amino acid residue 319-545.

5. The method of claim 1, wherein the recombinant polypeptide consists essentially of amino acid residue 319-545 within the RBD of SARS-CoV-2 spike protein.

6. The method of claim 1, wherein said SARS-CoV-2 spike protein is a SARS-CoV-2 S1 protein.

7. The method of claim 1, wherein the recombinant polypeptide has an average molecular weight of more than 28 kDa.

8. The method of claim 1, wherein the recombinant polypeptide has an average molecular weight of from about 28 kDa to about kDa.

9. The method of claim 1, wherein the recombinant polypeptide has an average molecular weight of about 34 kDa.

10. The method of claim 1, wherein the recombinant polypeptide comprises a plurality of N-glycosylation sites.

11. The method of claim 1, wherein the recombinant polypeptide comprises 17 glycan moieties on N331.

12. The method of claim 1, wherein the recombinant polypeptide comprises 12 glycan moieties on N334.

13. The method of claim 1, wherein the recombinant polypeptide comprises 19 glycan moieties on N343.

14. The method of claim 1, wherein the recombinant polypeptide comprises a plurality of O-glycosylation sites.

15. The method of claim 14, wherein the O-glycosylation sites comprise seven serine residues (S366, S371, S373, S375, S438, S443 and S514).

16. The method of claim 14, wherein the O-glycosylation sites comprise three threonine residues (T333, T376 and T523).

17. The method of claim 1, further comprising co-administering to the primate an immunologic adjuvant.

18. The method of claim 17, wherein the immunologic adjuvant is selected from the group consisting of aluminum salts, Toll-Like-Receptor (TLR) agonist, oil-in-water emulsion adjuvants, saponin-based adjuvants, and combination thereof.

19. The method of claim 17, wherein the immunologic adjuvant is aluminum hydroxide.

20. The method of claim 1, wherein the immune response against SARS-CoV-2 induced by the effective amount of the recombinant polypeptide blocks SARS-CoV-2 infection in the primate by at least about 60%, at least about 65%, at least about 70%, at least about 75%, at least about 80%, at least about 85%, at least 90%, at least 95%, at least 96%, at least about 97%, at least about 98%, or at least 99%.

21.-43. (canceled)
